(19) United States
(12) Patent Application Publication
(10) Pub. No.: US 2025/0318742 A1
(43) Pub. Date: Oct. 16, 2025

(54) IMPEDANCE MEASUREMENT AND TUNING USING CIRCULATORS, AND RELATED METHODS

(71) Applicant: JRE Star Investment Holdings, LLC, Farmington, UT (US)

(72) Inventors: Sharisse A. Poff, Draper, UT (US); Marcus Frederick Julian, Eden, UT (US); Shiuh-hua Wood Chiang, Vineyard, UT (US); Robert C. Davis, Provo, UT (US)

(21) Appl. No.: 19/094,783

(22) Filed: Mar. 28, 2025

Related U.S. Application Data
(60) Provisional application No. 63/575,325, filed on Apr. 5, 2024.

(51) Int. Cl. A61B 5/0535

(52) U.S. Cl. CPC A61B 5/0535

(57) ABSTRACT

Methods and systems for tuning impedance measurements, such as bioimpedance measurements. In some methods, an input signal may be generated and sent through a circulator, such as an active quasi-circulator. The input signal may be passed through a portion of a human body, such as a blood vessel, and back into the circulator. The input signal may be tuned to increase the sensitivity of the output signal to a pulsatile component of the signal, such as a portion of the signal associated with a heartbeat.

### CROSS-REFERENCE TO RELATED APPLICATIONS

This application claims the benefit under 35 U.S.C. § 119 (e) of U.S. Provisional Patent Application No. 63/575,325, which was filed Apr. 5, 2024, and titled “A 25-MHz 25-mW 0.012-mm2 Integrated Active Quasi-Circulator for Impedance Measurement,” which is hereby incorporated herein by reference in its entirety.

### SUMMARY

Bioimpedance is the property of living tissue to oppose the flow of electric current. Calculation of bioimpedance is typically done using Ohm's Law. An electric current is injected into the tissue, and the resulting voltage drop is measured. The complex valued impedance is then calculated by taking the ratio of the voltage over the current. The quantity can be expressed either in terms of the real and imaginary part of the impedance (resistance and reactance) or the magnitude and phase of the impedance.

Bioimpedance measurements can be used to determine a variety of metrics regarding the status of a body. Some applications of bioimpedance measurements include body composition, hydration, blood pressure, and blood glucose.

Due to the dynamic physiological changes that occur in the human body, bioimpedance signals constantly change over time. Various processes, such as breathing and movement, may modulate a bioimpedance signal in either a regular or irregular manner.

One modulation source of particular interest is related to blood flow and the heart, as shown in FIG. 1. During the course of a heartbeat, the change between systolic and diastolic pressure causes arteries and veins to expand and contract. This expansion and contraction causes a change in blood volume in blood vessels. Since the blood has a higher conductivity than muscle and fat, as blood volume increases during the systolic phase, the overall measured impedance decreases, and vice versa during the diastolic phase. Bioimpedance plethysmography (BIP) measures these volumetric changes in bodily fluids using impedance. BIP has been demonstrated for a variety of applications, including heart rate detection, pulse wave velocity, and blood glucose monitoring. For blood glucose in particular, plethysmographic measurements are needed in order to separate arterial blood signals, which contain stronger glucose information, from interstitial fluid signals.

Existing BIP systems typically measure at kHz excitation frequencies. However, MHz excitations are of interest for certain blood monitoring applications, such as glucose monitoring, because the effects of electrode polarization, which should be minimized, decrease as frequency increases. Because of the benefits of plethysmographic measurements to measure arterial blood independently from interstitial fluid, BIP offers a promising avenue for increased correlation with blood glucose.

MHz BIP measurement devices are not readily available, leaving BIP largely unexplored at these frequencies. A compact MHz BIP system would enable researchers to study glucose and other properties of the blood at MHz frequencies, thereby providing a plethora of research opportunities in noninvasive bioimpedance monitoring.

Impedance measurement has applications in living tissue characterization, material analysis, and mechanical structure monitoring, among others. These applications benefit from impedance spectroscopy-measurements across a frequency range-because properties of a device-under-test (DUT) respond differently to different excitation frequencies, thus revealing the details of the material's makeup. This concept applies to the measurement of living tissues, where low frequencies travel around cells and high frequencies travel through cell membranes. Cell properties also vary across dispersions (α, β, δ, γ), resulting in frequency-dependent measurement results. In addition to a range of frequencies, tissue measurement applications also benefit from high sensitivity. Increased sensitivity effectively magnifies changes in impedance, which is especially valuable for the measurement of the relatively small pulsatile impedances found in, e.g., heart-rate monitoring and biological property characterization for medical diagnosis and treatment.

For living tissue measurements, benchtop equipment such as vector network analyzers (VNAs) and lock-in amplifiers can operate over very wide bandwidths, but they are large and power-hungry—unsuitable for continuous monitoring wearables—as well as sensitivity-limited. While integrated circuits have been demonstrated for wearable impedance measurement, especially for bioimpedance applications, they are mostly limited to kHz or lower excitation frequencies. Works operating above 10 MHz employ pre-demodulation techniques. Some of these precede the demodulation choppers with broadband amplifiers to attenuate chopper kickback. Impedance measurement at frequencies exceeding 10 MHz is valuable, however, for measuring certain physiological changes, e.g., bioimpedance changes due to blood glucose levels, which have been shown to produce a strong impedance response from 40-120 MHz and 27-57 MHz. Thus, a simple circuit which operates at MHz frequencies with high sensitivity would be valuable in this field.

A significant barrier to achieving MHz-range impedance measurement circuit frequency is the issue of parasitic capacitance. While op amps in designs such as the 4-point measurement circuit present high input impedance to kHz excitations, the increased effects of parasitic capacitance significantly lower the input impedance with MHz excitations, rendering the designs impractical. This barrier may, in some instances, be overcome with a circulator, such as a quasi-circulator in some cases, combined with resonance techniques, to extend circuit bandwidth. Traditionally used in RF communications, circulators have a unique advantage of controlled port impedance (typically 50Ω), which can enable high frequency impedance measurement by minimizing the effects of parasitic capacitance.

A subset of circulator, the active quasi-circulator (AQC), can be integrated, making it a good candidate for wearable devices and/or applications. One challenge of AQCs, however, is that they provide high sensitivity only in cases where the impedance of the DUT is close to their port impedance. Where the impedances differ, measurement sensitivity degrades. A circulator with tunable port impedance may address this issue, but this technique is previously unexplored.

To overcome the challenges of size, frequency range, and sensitivity, the present inventors propose an impedance-measurement circuit utilizing, in preferred embodiments, an active quasi-circulator (AQC), which presents a unique technique for wearable applications, such as wristbands and the like. The AQC approach, in preferred implementations, is unique among impedance measurement circuits in at least three main ways: 1) its integrated structure lends itself to wearables, 2) through controlled port impedance, it enables measurements at higher frequencies, such as frequencies above 1 MHz in some cases, than high-impedance bioimpedance measurement circuits and at frequencies comparable to pre-demodulation circuits without the need for choppers, and 3) through real-time-tunable impedance matching, it translates signals of interest to very low amplitudes so they can be substantially amplified without saturation, thus increasing measurement sensitivity. Some embodiments of impedance measurement circuits disclosed herein can be realized in a CMOS process, such as a 180-nm CMOS process; some may demonstrate high sensitivity at an excitation frequency of, for example, up to or even beyond 25 MHz; and/or some may, in some cases, occupy an active area as small as about 0.012 mm², thereby facilitating use in connection with wrist/radial artery sensors and the like.

Measurement results show that the proposed AQC design is a promising solution for sensitive measurement of DUT impedance in the MHz excitation range for wearables, thus providing an avenue for applications such as high-frequency blood glucose monitoring and the like.

In various embodiments and implementations disclosed herein, BIP measurements can be performed at MHz excitation frequencies. In preferred embodiments, the system utilizes an active quasi-circulator, a gain/phase detector, frequency generation, and analog-to-digital converters to measure bioimpedance, with the core circuits preferably fitting within the size of a wrist-worn device. We demonstrate the system's ability to dynamically optimize measurement sensitivity to human pulsatile bioimpedance signals. This application shows the system's capability for MHz BIP, a unique research field with promising potential for blood glucose monitoring. Additionally, our human subject test results show excellent sensitivity compared to conventional PPG measurement, making this technique, combined with or replacing a PPG signal, a promising avenue for noninvasive health applications beyond noninvasive glucose monitoring alone, such as arrhythmia detection, apnea monitoring, and oximetry verification.

Disclosed herein are therefore various methods, devices, and/or systems for performing impedance measurements, such as bioimpedance plethysmography measurements, preferably using an active quasi-circulator. Some embodiments may be designed to deliver an input signal and take measurements above 1 MHZ, and in some cases the sensitivity to small impedance changes can be dynamically optimized through the quasi-circulator match point. Such bioimpedance measurements may be useful for determining a variety of biological conditions, such as blood glucose levels or other blood analyte conditions. By accounting for small impedance variations due to the arterial pulse, for example, the effect of the blood can be studied more closely. The methods and systems presented herein may enable monitoring of pulsatile impedance changes with high input frequencies. Again, in some cases, such input frequencies may be above 1 MHz, or even higher, such as above 5 MHZ, above 8 MHZ, or above 10 MHz in some cases.

The adjustable R_{match}, as described below, may allow for dynamic tuning of the system to increase sensitivity. By tuning R_{match} to be near the match point of the system, the pulsatile signal in the output magnitude can be amplified, and tuning R_{match} to be at the match point causes the pulsatile signal in the output phase to be amplified.

Bioimpedance plethysmography (BIP) enables correlation of measurements with properties of human tissue. Some biological properties, such as blood glucose, exhibit characteristics measurable at MHz excitation frequencies, especially through BIP. Existing BIP systems, however, measure with kHz excitations. This disclosure proposes a system for performing BIP measurements, in some cases at MHz excitation frequencies, and the system is tested by measuring human arterial pulse. This application shows the system's ability to perform MHz BIP with dynamically-optimizable sensitivity, thus providing an avenue for future research on the response and correlation of various blood properties to MHz excitation.

In a more specific example of a method for tuning an impedance measurement system, such as a bioimpedance measurement system, according to some implementations, the method may comprise generating an input signal. In some cases, the input signal may comprise an alternating current signal having a frequency above 1 MHz. In some such cases, the frequency may be above 10 MHz.

The input signal may be sent into and/or through a circulator, such as an active quasi-circulator in some cases. In some cases, the input signal may be sent through a port, such as a first port, of the quasi-circulator or other circulator.

A load may then be applied to a port, such as the first port or a second port in some cases, of the quasi-circulator or other circulator.

The input signal may be tuned towards a match point of the quasi-circulator or other circulator. In some cases, this tuning may be performed to increase sensitivity of an output signal to impedance changes in the load.

In some implementations, the impedance measurement system may be non-invasive.

In some implementations, the impedance measurement system may be an impedance spectroscopy system.

In some implementations, the impedance measurement system may be a bioimpedance spectroscopy system.

In some implementations, the impedance measurement system may be an impedance plethysmography system.

In some implementations, the impedance measurement system may be a bioimpedance plethysmography system.

In some implementations, the impedance measurement system may be a dielectric measurement system.

In some implementations, the impedance measurement system may be a dielectric spectroscopy system.

In some implementations, the impedance measurement system may be a blood measurement system.

In some implementations, the step of tuning the input signal may comprise increasing sensitivity of the output signal to a pulsatile signal and/or pulsatile component of an output signal, such as a heartbeat.

In some implementations, the load may comprise a portion of a human body, such as a blood vessel.

In some implementations, the step of tuning the input signal towards a match point of the quasi-circulator may comprise tuning the input signal to the match point.

In some implementations, the step of tuning the input signal towards a match point of the circulator may comprise tuning the signal to enable extraction of an impedance value through analysis of a circulator transfer function.

In some implementations, the match point of the circulator may be a point at which the magnitude of the output signal is zero, or at least substantially zero.

In an example of a method for increasing sensitivity to a pulsatile signal in an impedance measurement system, such as a bioimpedance measurement system, according to some implementations, the method may comprise generating an input signal, such as an alternating current input signal that may, in some cases, be a high-frequency signal above 1 MHz.

The input signal may be sent into and/or through a circulator, such as an active quasi-circulator. In some cases, the input signal may be sent into and/or through a first port of the circulator.

The input signal may then be passed through a portion of a human body, such as a blood vessel. In some cases, the input signal may be passed from and/or through the first port, or a second port, of the circulator.

Following the step of passing the input signal from the circulator through a portion of a human body, the input signal may be output from the circulator, such as output at and/or through the first port, the second port, or a third port of the circulator.

The input signal may then be tuned to increase a pulsatile component of an output signal.

In some implementations, the step of tuning the input signal may comprise adjusting an impedance associated with the input signal towards a match point of the quasi-circulator. In some such cases, the step of tuning the input signal may comprise adjusting an impedance associated with the input signal to, or at least substantially to, the match point of the quasi-circulator.

In some implementations, the match point of the quasi-circulator or other circulator comprises a point at which a magnitude of the output signal is zero, such as zero volts.

In some implementations, the pulsatile component may be generated by a heartbeat.

In some implementations, the pulsatile component may be plethysmographic.

In an example of a system for tuning impedance measurements, such as bioimpedance measurements, to increase sensitivity to pulsatile signals, according to some embodiments, the system may comprise an alternating current generator configured to generate an input signal, which may in some embodiments be configured to be sent into a human body. In some cases, the input signal may be at least 1 MHZ, or at least 10 MHz in some such cases.

The system may further comprise a circulator, such as a quasi-circulator (in some cases, an active quasi-circulator) that is configured to receive the input signal and output an output signal, in some cases after the input signal has passed through the human body.

The system may further comprise a tuning module configured to adjust a component of the input signal to increase sensitivity of the output signal to pulsatile impedance changes.

In some embodiments, the tuning module may be configured to adjust an impedance associated with the input signal. In some such embodiments, the tuning module may be configured to digitally adjust the impedance.

In some embodiments, the tuning module may be configured to increase sensitivity of the output signal to pulsatile impedance changes by tuning the input signal towards a match point of the quasi-circulator. In some cases, the tuning module may be configured to increase sensitivity of the output signal to pulsatile impedance changes by tuning the input signal to the match point of the quasi-circulator.

In some embodiments, the match point of the quasi-circulator may be reached when the output signal of the quasi-circulator has a magnitude of zero, such as zero volts.

Some embodiments may further comprise a safety circuit configured to limit the magnitude of the current entering the human body and/or prevent or at least inhibit voltage spikes.

In another example of a method for extracting an impedance value using an impedance measurement system according to some implementations, the method may comprise sending an input signal into a first port of a circulator, such as a quasi-circulator. Again, the input signal may comprise an alternating current signal, in some cases an alternating current signal having a frequency above 1 MHZ. A load may then be applied to a second port of the quasi-circulator. In some cases, the load may comprise a portion of the human body. The input signal may then be tuned to a match point of the quasi-circulator to enable extraction of an impedance value through analysis of the quasi-circulator transfer function.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 depicts an arterial pressure waveform with the dicrotic notch demarcating the transition between the systole and diastole phases, along with the impedance variation due to blood volume changes;

FIG. 2A depicts an ideal circulator showing signal flow between the circulator ports;

FIG. 2B depicts a quasi-circulator showing how signal flow differs from an ideal circulator and showing how the path from P3 to P1 is eliminated and an out-of-phase path is added to cancel the P1-to-P3 feedthrough;

FIG. 2C depicts a conceptual circuit implementation comprising a quasi-circulator for impedance measurement with the small-signal output determined by two out-of-phase transconductors and a load resistor;

FIG. 3 is a circuit diagram for impedance measurement using an active quasi-circulator with the right path implemented as a common source amplifier with Gₘ (Gₘ₁ in FIG. 2C) varying with the DUT (2×Z_{L}), the left path implemented as a common gate amplifier with Gₘ (Gₘ₂ in FIG. 2C) varied by R_{V};

FIG. 4 is an active quasi-circulator small signal model used to analyze the transfer function and extract the value of Z_{L} with port locations labeled and with the DC blocking capacitors depicted following ΔVᵢₙ to show that P1 is split into P1_{cg} and P1_{cs} due to the difference in DC bias voltage;

FIG. 5A is a graph illustrating example Vₒᵤₜ simulated transient waveforms at 10 MHz resonance for difference R_{V} values with R_{L}=50Ω to illustrate how magnitude and phase change with R_{V};

FIG. 5B is a graph illustrating Vₒᵤₜ magnitudes: model v. simulation at 10 MHZ resonance for various R_{L} values with the nulls in the magnitude showing the value of R_{V} for each R_{L} value that results in Gₘ1=−Gₘ₂;

FIG. 5C is a graph illustrating the sensitivity at 10 MHz resonance (in dB/Ω) showing maxima immediately before and after the Vₒᵤₜ magnitude nulls;

FIG. 6A is a diagram of a fabricated die;

FIG. 6B is a circuit diagram showing an experimental setup, including the signal generator (Agilent 33250A), input buffer (ADA4937) and associated resistors (R_{T}, R_{S}, R_{G}, R_{F}), DC blocking capacitors (C₁), and potentiometers (R_{V}) at P1; at P2, DC blocking capacitors (C₂), resonant components (Zᵣ), and DUT (C_{L}, R_{L}); at P3, DC blocking capacitors (C₃) and oscilloscope (Keysight DSOS204A);

FIG. 6C shows the physical testbench of the experimental setup with oscilloscope, signal generator, power supply, PCB and AQC chip;

FIG. 7A is a graph showing Vₒᵤₜ magnitudes with resonant frequency, f=1/(2π∞ZᵣC_{L}), for various R_{L} values at R_{V} match with C_{L} and Zᵣ resonant at 10 MHZ;

FIG. 7B is a graph showing Vₒᵤₜ magnitudes with resonant frequency f=1/(2π∞ZᵣC_{L}), for various R_{L} values at R_{V} match with C_{L} and Zᵣ resonant at 25 MHZ;

FIG. 8A is a graph showing Vₒᵤₜ magnitude versus R_{V} for different R_{L} values at the frequency nulls found in the previous plots at 10 MHz resonance;

FIG. 8B is a graph showing Vₒᵤₜ magnitude versus R_{V} for different R_{L} values at the frequency nulls found in the previous plots at 25 MHz resonance;

FIG. 9 is a diagram showing the output behavior of a quasi-circulator used in various embodiments and/or examples presented herein;

FIG. 10A is a graph showing the output behavior of an ideal quasi-circulator vs. measured voltage output with varying values of R_{DUT};

FIG. 10B is a graph showing the output behavior of an ideal quasi-circulator vs. measured phase output with varying values of R_{DUT};

FIGS. 11A and 11B are diagrams showing the architecture of a quasi-circulator system according to some embodiments;

FIG. 12 is a schematic diagram of safety circuitry that may be used for a safety circuit in some embodiments; this figure shows components used both for user/human isolation and protection against large voltage spikes;

FIG. 13 illustrates an example of a quasi-circulator system PCB according to some embodiments;

FIG. 14 is a schematic diagram illustrating an optocoupler circuit according to some embodiments, which figure indicates that, by switching a relatively large R_{P2} in and out of the circuit, the total resistance can be modulated by a small amount;

FIG. 15A is a graph showing output pulse magnitude amplitudes of a quasi-circulator system according to some embodiments with an optocoupler load at port 2 of the quasi-circulator for various values of R_{match},

FIG. 15B is a graph showing output phase responses of a quasi-circulator system according to some embodiments with an optocoupler load at port 2 of the quasi-circulator for various values of R_{match};

FIG. 16 is a testbench of a system setup for human measurements according to some embodiments;

FIGS. 17A and 17B are graphs of output pulses in magnitude and phase for a first participant in a working example for various values of R_{match};

FIGS. 17C and 17D are graphs of output pulses in magnitude and phase for a second participant in a working example for various values of R_{match};

FIG. 18 is a graph plotting the magnitude output for a participant in a working example with key cardiac cycle points labelled;

FIGS. 19A and 19B are graphs comparing PPG signals and Quasi-Circulator System Phase signals over time for a participant in a working example with R_{match} set to the match point of 160Ω; and

FIGS. 20A and 20B are graphs comparing the magnitude and phase signal amplitudes for each participant in a working example.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

A DUT with impedance Z_{L} can be modeled, without loss of generality, by a resistive part (R) in series with a reactive part (X). In some examples provided herein, the former is determined using matching and the latter using resonance, as described next. The results are then added together (Z=R+jX) to provide a complete impedance measurement. To find the value of R, the matching approach works as follows:

An input signal is sent through a conceptual circuit that produces a reflected signal whose magnitude depends on the value of R. If an input signal of the same magnitude and opposite phase is added to this reflected signal, the output of the circuit will be zero (i.e., nulled), and the circuit's transfer function can be used to determine the value of R. This can be achieved by tuning a resistor in the reflected signal's path. This conceptual circuit can be realized using the AQC topology explained later. In the proposed AQC design, a known tuning resistance and a known transfer function are used to extract the resistance of the DUT.

To find the value of X, the resonance approach works as follows: X is modeled as either capacitive (X_{C}=−1/(2πfC)) or inductive (X_{L}=+2πfL), where f represents the excitation frequency. Because these impedances have opposite signs, a capacitor in series with an inductor, satisfying the equation fᵣₑₛ=1/(2πVLC), has zero impedance at a single resonant frequency fᵣₑₛ. At this frequency, the impedance of the DUT becomes purely real (e.g., R_{L}), producing a null in the circuit's output magnitude. In the proposed AQC design, the reactive impedance of the DUT is negative, so a known inductance and a known resonant frequency may be used to extract the reactance.

Considering these techniques in relation to each other, resonance produces a minimum magnitude at a single frequency (i.e., a null in a frequency sweep), and matching produces a minimum magnitude at a single tuning resistance (i.e., a null in a tuning-resistance sweep). Both resonance and matching together bring the null to zero volts. In the proposed AQC design, the lower the null, the steeper the slope immediately surrounding it.

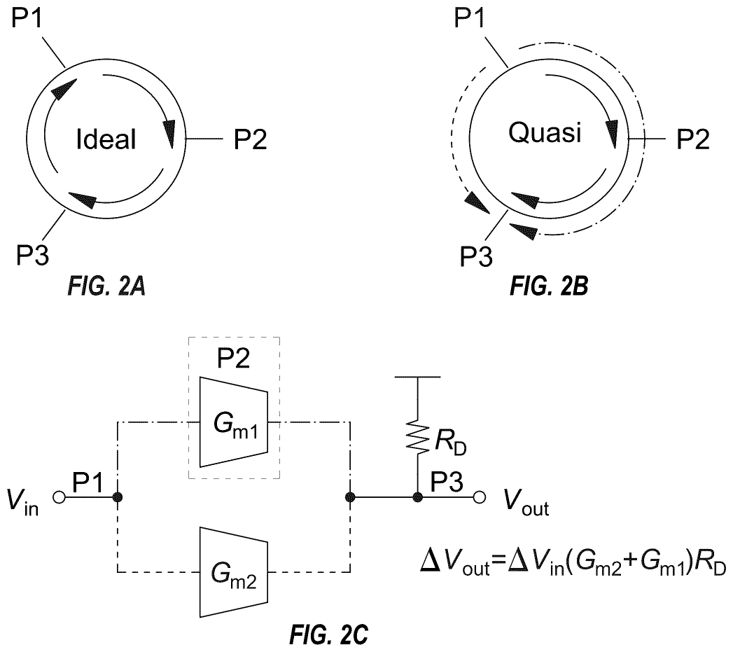

A circulator is a multi-port non-reciprocal device which allows signals to flow between ports in one direction and blocks signals from flowing in the reverse direction. In the three-port circulator shown in FIG. 2A, for example, signals flow clockwise from port 1 (P1) to port 2 (P2), from P2 to port 3 (P3), and from P3 to P1, while no signal flows in the counterclockwise direction.

A circulator can be a passive or active device. Passive circulators achieve non-reciprocity using ferrite materials and applied magnetic fields, while active circulators are implemented with inherently non-reciprocal transistors. The need to supply power is a disadvantage for active circulators, but they are advantageous over passive circulators for minimal chip area and dynamic design control.

A quasi-circulator is similar to a circulator, but it eliminates the path from P3 to P1. An additional path is often added from P1 to P3, designed to send a signal that is 180 degrees out of phase with the signal sent from P1 to P2. This path improves the isolation between P1 and P3 by cancelling the signal from P1 that makes its way through P2 to P3. This concept is depicted in FIG. 2B.

In this figure, the two signals from P1 are 180 degrees out of phase with each other, so when they both arrive at P3, they cancel each other out, leaving only the signal from P2 to be measured at P3.

An implementation of the quasi-circulator concept is depicted in FIG. 2C. The input signal (Vᵢₙ) travels from P1 through two transconductors (Gₘ₁ and Gₘ₂). P2 is internal to Gₘ₁, so it is implicit in the diagram. The outputs from the Gₘ blocks are added together, and the resulting signal is converted from current to voltage by the resistor (R_{D}), producing the P3 output signal (Vₒᵤₜ). The transconductors are out of phase from each other, so P1-to-P3 feedthrough can be cancelled. The small-signal input and output are ΔVᵢₙ and ΔVₒᵤₜ, respectively.

The quasi-circulator can be thought of in microwave terms as a device that guides incident and reflected signals. P2 sees an incident signal from P1 and sends a reflected signal to P3. If a circuit placed at P2 presents a resonant impedance match with the port, the magnitude of the signal measured at P3 will be zero. This nulling functionality, similar to that of the Wheatstone bridge, is the basis of some embodiments of the proposed AQC design.

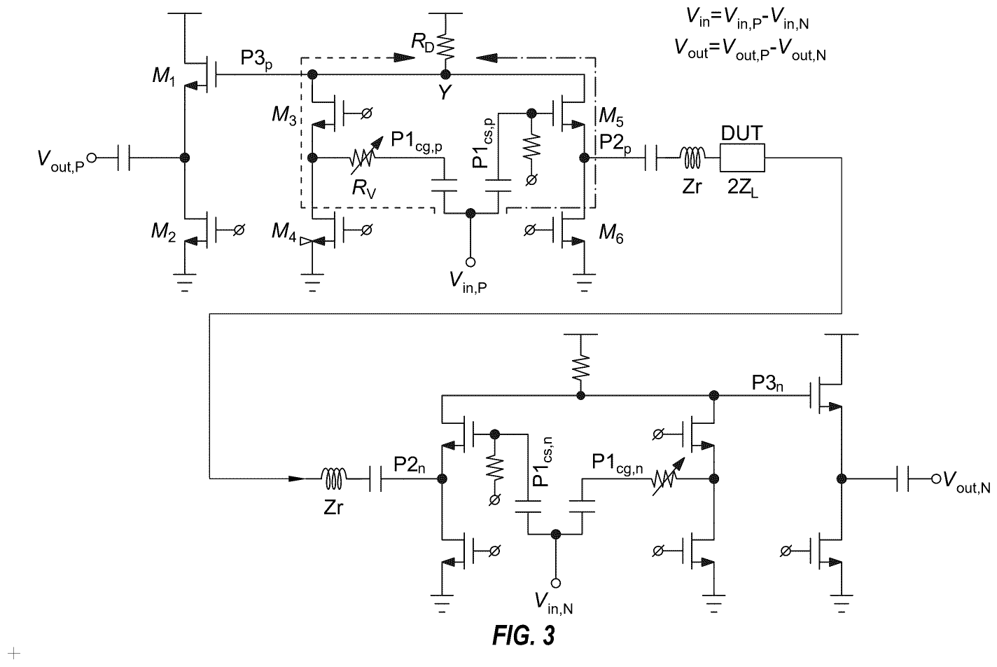

The transistor-level implementation of some embodiments of the AQC design can be seen in FIG. 3. This topology may be used for impedance measurement to overcome frequency limitations faced by the prior art. A voltage waveform (V_{in,p}) enters the chip, and DC blocking capacitors separate the signal into two DC bias voltages (P1_{cg,p} for common gate bias and P1_{cs,p} for common source bias). At P1_{cs,p}, the signal V_{in,p} travels through the lower transconducting path, which may be implemented as a common source amplifier formed by M₅ and M₆.

The DUT is placed at P2ₚ, which is at the source of M₅. At P1_{cg,p}, V_{in,p} travels through the upper transconducting path, which may be implemented as a common gate amplifier formed by M₃ and M₄. The source impedance of this amplifier (R_{V}) is variable. At P3ₚ, the common gate output is added to the common source output, and the resulting signal is converted from current to voltage by a resistor (R_{D}). The combined signal is buffered by a common drain amplifier, which may be formed by M₁ and M₂, and exits the chip as V_{out,p}. M₂, M₄, and M₆ may be implemented as current mirrors, with current set by a diode-connected transistor.

The impedance looking into the amplifiers' sources is designed to be 50Ω. This presents 50-Ω impedance to the DUT at P2ₚ and to a measurement device at P3ₚ. The input impedance is infinite at P1_{cs,p} and variable (due to the variable resistor R_{V}) at P1_{cg,p}, so an off-chip buffer is added to buffer this impedance and present 50-Ω impedance to a signal generator. The design may be implemented differentially to suppress common-mode noise: A signal 180 degrees out of phase from V_{in,p} (labelled V_{in,n}) travels through an identical circuit to V_{out,n}. The component Zᵣ is an off-chip inductor in series with the DUT, which has a value of 2×Z_{L} due to the differential design. All capacitors are 10 μF for DC blocking.

A null can be produced in the AQC output of FIG. 3 by setting Zᵣ to resonate with the reactive impedance of 2×Z_{L} at the excitation frequency and setting R_{V} so that Gₘ₁=−Gₘ₂.

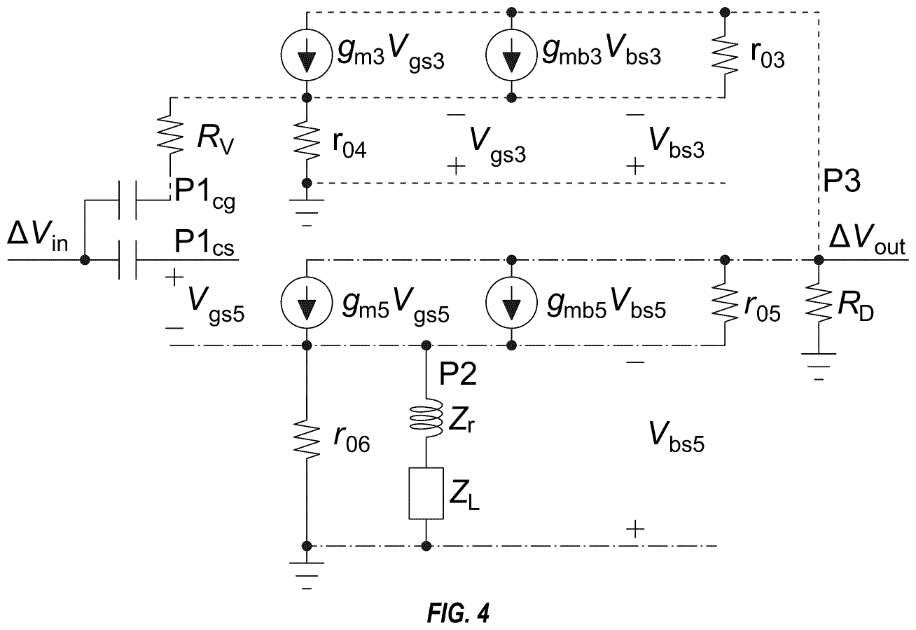

The transconductances Gₘ₁ and Gₘ₂ depicted in FIG. 2C can be mapped to the common source amplifier and common gate amplifier, respectively, and defined using small-signal parameters. A small-signal model of the AQC is shown in FIG. 4. Ignoring channel-length modulation and body effect to show the quasi-circulator cancellation technique in simple terms, the transconductance of the lower path is:

$${G}_{m 1} {\vert}_{{r}_{0} = ∞ , {g}_{mb} = 0} = - {g}_{m 5} / ( 1 + {g}_{m 5} ( {Z}_{L} + {Z}_{r} ) )$$

where Z_{L} represents the DUT in series with a resonant component Zᵣ. Similarly, the transconductance of the upper path is:

$${G}_{m 2} {\vert}_{{r}_{0} = ∞ , {g}_{mb} = 0} = {g}_{m 3} / ( 1 + {g}_{m 3} {R}_{V} )$$

where R_{V} represents a variable resistor.

The small-signal output ΔVₒᵤₜ is the transconductance multiplied by the load resistance (R_{D}):

$${ΔV}_{out} {\vert}_{{r}_{0} = ∞ , {g}_{mb} = 0} = {ΔV}_{i n} ( {G}_{m 2} + {G}_{m 1} ) {R}_{D}$$

Substituting Gₘ₁ and Gₘ₂, ΔVₒᵤₜ becomes:

$${ΔV}_{out} {\vert}_{{r}_{0} = ∞ , {g}_{mb} = 0} = {ΔV}_{i n} ( \frac{{g}_{m 3} {R}_{D}}{1 + {g}_{m 3} {R}_{V}} - \frac{{g}_{m 5} {R}_{D}}{1 + {g}_{m 5} ( {Z}_{L} + {Z}_{r} )} )$$

ΔVₒᵤₜ is subsequently buffered by a source-follower (M₁ and M₂) to drive off-chip data acquisition. The magnitude of ΔVₒᵤₜ may be determined by: 1) the difference between the frequency (f) of Vᵢₙ and the resonant frequency (fᵣₑₛ) of Zᵣ with Z (the magnitude is smallest (i.e., nulled) when f=fᵣₑₛ) and 2) the amount of mismatch seen between Z_{L}+Zᵣ and R_{V} (the magnitude is smallest (i.e., nulled) when Z_{L}+Zᵣ=R_{V}).

The AQC transfer function is based on the equation above, which ignores channel-length modulation and body effect for clarity in visualizing the AQC behavior. Such assumptions do not hold for accurate impedance calculations, so these terms are included when calculating Vₒᵤₜ model magnitudes. Derived from the small-signal model in FIG. 4, the full equation for output magnitude is:

$${ΔV}_{out} {\vert}_{{r}_{0} \neq ∞ , {g}_{mb} \neq 0} = {ΔV}_{i n} ( \frac{1 + {σ}_{2} / {σ}_{3}}{{R}_{V}} + \frac{{g}_{m 3} {σ}_{2}}{{σ}_{3}} + \frac{{g}_{mb 3} {σ}_{2}}{{σ}_{3}} + \frac{{σ}_{2}}{{r}_{03} {σ}_{3}} + \frac{{σ}_{2}}{{r}_{03} {σ}_{3}} ) / ( \frac{{σ}_{1} / {σ}_{3} + 1}{{r}_{03}} + \frac{{σ}_{1}}{{r}_{04} {σ}_{3}} + \frac{{σ}_{1}}{{R}_{V} {σ}_{3}} + \frac{{g}_{m 3} {σ}_{1}}{{σ}_{3}} + \frac{{g}_{mb 3} {σ}_{1}}{{σ}_{3}} ) \qquad ( 2 )$$

$$where$$

$${σ}_{1} = \frac{1}{{r}_{05}} ( \frac{1}{{r}_{05} {σ}_{4}} - 1 ) - \frac{1}{{R}_{D}} - \frac{1}{{r}_{03}} + \frac{{g}_{m 5}}{{r}_{05} {σ}_{4}} + \frac{{g}_{mb 5}}{{r}_{05} {σ}_{4}} \qquad ( 3 )$$

$${σ}_{2} = \frac{{g}_{m 5}}{{r}_{05} {σ}_{4}} + {g}_{m 5} ( \frac{{g}_{m 5}}{{σ}_{4}} - 1 ) + \frac{{g}_{m 5} {g}_{mb 5}}{{σ}_{4}} \qquad ( 4 )$$

$${σ}_{3} = {g}_{m 3} + {g}_{mb 3} + \frac{1}{{r}_{03}} \qquad ( 5 )$$

$${σ}_{4} = {g}_{m 5} + {g}_{mb 5} + \frac{1}{{r}_{05}} + \frac{1}{{r}_{06}} + \frac{1}{{Z}_{L} + {Z}_{r}} . \qquad ( 6 )$$

Using the resonance and matching approach, the output of the transfer function is zero for a resonant, matched circuit. For that case, ΔVₒᵤₜ/ΔVᵢₙ=0 and Z_{L}+Zᵣ=R_{L}, resulting in an equation solvable for the resistive part of the DUT:

$${R}_{L} = {[ - {g}_{m 5} - {g}_{mb 5} - \frac{1}{{r}_{05}} - \frac{1}{{r}_{06}} + ( {g}_{m 5} + {g}_{mb 5} + \frac{1}{{r}_{05}} ) ( {g}_{m 3} + {g}_{mb 3} + \frac{1}{{r}_{03}} + \frac{1}{{r}_{04}} + \frac{1}{{R}_{V}} ) / ( {g}_{m 3} + {g}_{mb 3} + \frac{1}{{R}_{V}} - \frac{{g}_{m 3}}{{g}_{m 5} {R}_{V}} - \frac{{g}_{mb 3}}{{g}_{m 5} {R}_{V}} - \frac{1}{{g}_{m 5} {r}_{03} {R}_{V}} + \frac{1}{{r}_{03}} + \frac{1}{{r}_{04}} ) ]}^{- 1} . \qquad ( 7 )$$

For small Vᵢₙ, this equation extracts R_{L} well, as demonstrated in the following section.

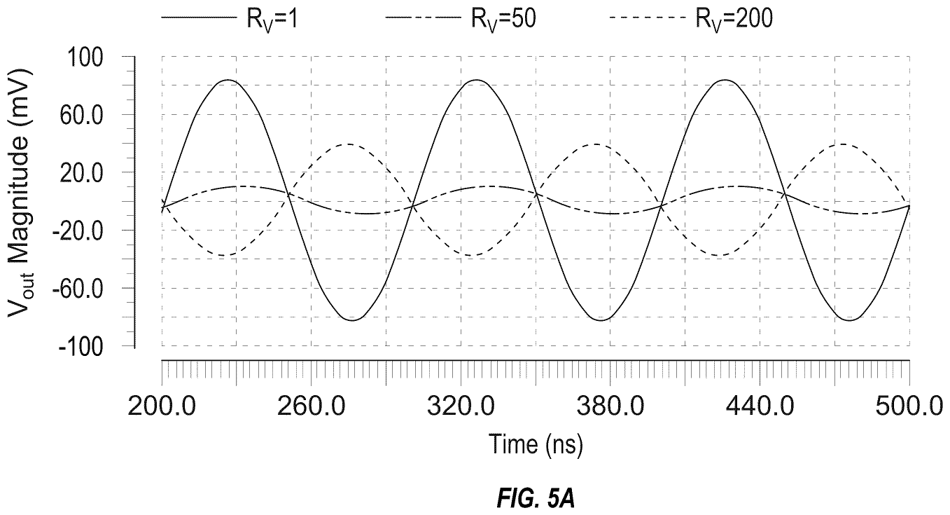

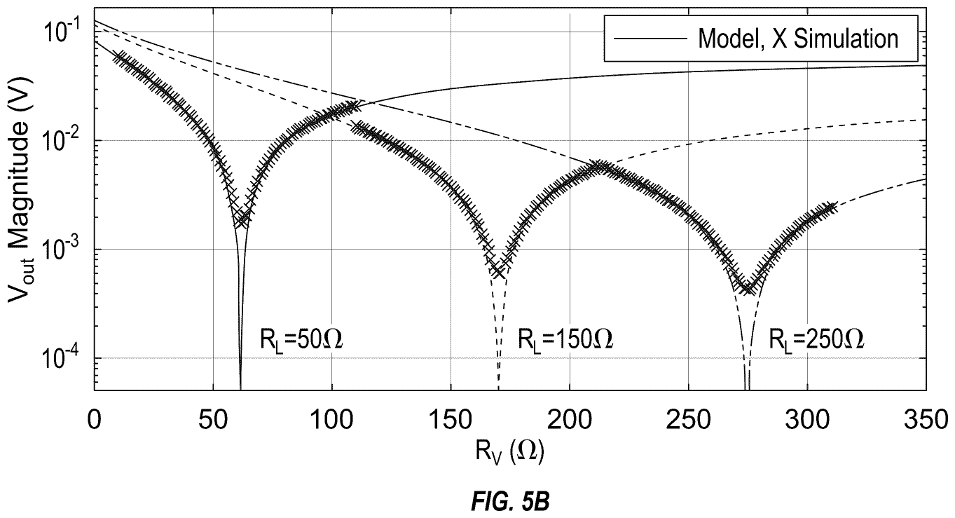

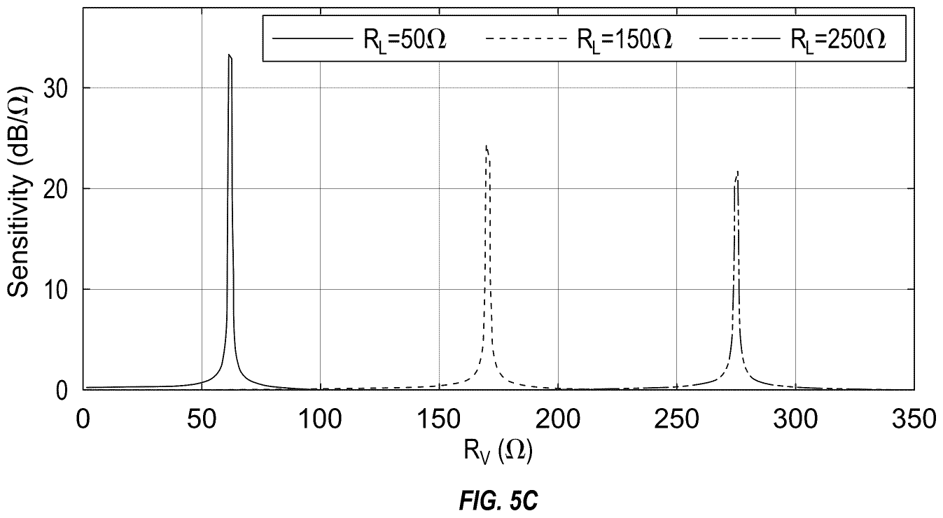

The transistor-level circuit is simulated, and FIG. 5A shows Vₒᵤₜ transient waveforms for different R_{V} values with Vᵢₙ magnitude set to 60 mVᵣₘₛ. When R_{V}=1Ω, Vₒᵤₜ magnitude is ˜80 mV, indicating |Gₘ₁| is much less than |Gₘ₂| (see FIG. 2). When R_{V}=50Ω, Vₒᵤₜ magnitude is ˜10 mV and Vₒᵤₜ phase has shifted by ˜10 degrees, indicating |Gₘ₁| is slightly less than |Gₘ₂|. When R_{V}=200Ω, Vₒᵤₜ magnitude is ˜40 mV, and Vₒᵤₜ phase has shifted an additional ˜165 degrees, indicating |Gₘ₁| is now greater than |Gₘ₂| and the magnitude null has been crossed. From Vₒᵤₜ transient waveforms such as these, the Vₒᵤₜ magnitudes are recorded on a logarithmic scale for various R_{V} values, shown in FIG. 5B.

As the analysis predicted, nulls are present at match (i.e., where Gₘ₁=−Gₘ₂). The simulated nulls are higher in voltage than the model nulls, likely because the model assumes perfect resonance, which the simulation does not achieve due to MOSFET parasitics. The R_{V} values associated with the minima are the same and the Vₒᵤₜ magnitudes for all but the nulls agree. This shows that the small-signal model describes the simulation well, though it will be shown later that the measurements differ from the model and simulation due to PCB parasitics and chip layout. These parasitics make the DUT complex, which affects the nulling functionality increasingly as frequency and R_{L} increase. The model and simulation can be calibrated to represent the measurement behavior, details of which are described further below.

The output is most sensitive to changes in the DUT value where the slope of Vₒᵤₜ magnitude versus R_{V} is greatest. Because the active quasi-circulator technique moves the signals of interest to the nulls through resonance and matching, Vₒᵤₜ can be substantially amplified without saturating the critical signals. This amplification can be done through conversion to the logarithmic domain, which amplifies small signals more than large signals due to the derivative relation (log₁₀(Vₒᵤₜ))′=1/(ln(10)Vₒᵤₜ). After the conversion, the greatest slope—and therefore the greatest sensitivity—is found immediately surrounding the magnitude nulls, exactly where the signals of interest lie. This sensitivity in dB/Q for the model Vₒᵤₜ magnitudes is depicted in FIG. 5C.

The highest sensitivity, which is theoretically infinite but limited by the step size of R_{V}, corresponds to the Vₒᵤₜ magnitude nulls. The sensitivity has a narrow peak and drops off sharply as R_{V} increases or decreases from the null. Due to the relationship of common-source degeneration (R_{L}) and common-drain gate resistance (R_{V}) to AQC gain, the circuit's sensitivity decreases as the resistive part of the DUT's impedance increases. This relation is seen by analyzing the partial derivative of the initial transfer function referenced above at resonance (where Z_{L}+Zᵣ simplifies to a purely resistive load R_{L}), as shown:

$$\frac{\partial }{\partial {R}_{L}} ( \frac{{g}_{m} {R}_{D}}{1 + {g}_{m} {R}_{V}} - \frac{{g}_{m} {R}_{D}}{1 + {q}_{m} {R}_{L}} ) = \frac{{g}_{m}^{2} {R}_{D}}{{( {g}_{m} {R}_{L} + 1 )}^{2}} \qquad ( 8 )$$

The AQC gain decreases with an inverse square relation to an increase in R_{L}: A∝1/R_{L}². Thus, greater sensitivity is achieved for low resistances, as seen in FIG. 5B and as demonstrated in the following section.

### Example 1

The proposed AQC was fabricated in 180-nm CMOS technology and mounted on a PCB. The circuit operates under a 1.8-V supply and consumes 25 mW. In our target application of measuring changes in living tissue for applications such as glucose monitoring, this power will be consumed intermittently (e.g., for a few seconds (specified by the SNR required for a given application) once every 10 minutes) because the bioimpedance changes on the timescale of minutes. This allows the circuit to be powered down most of the time, thus saving considerable power.

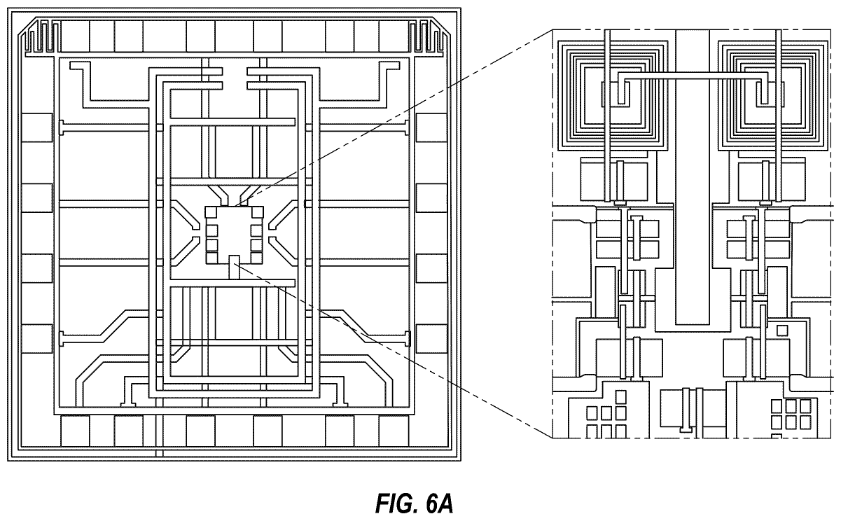

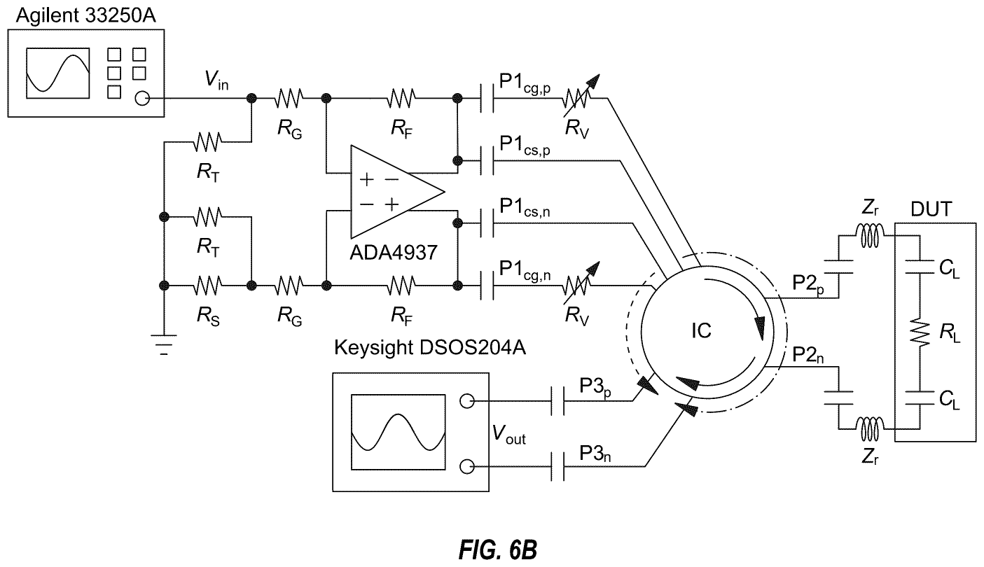

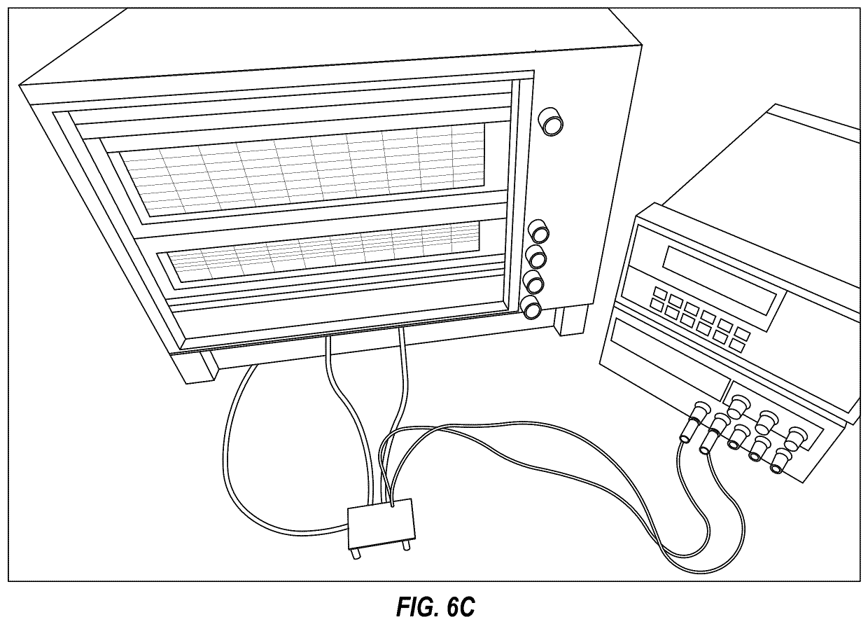

FIG. 6A shows a diagram of the fabricated die, including a zoomed-in image of the core with area 90 μm×128 μm=0.012 mm². The circuit is differential and therefore laid out with a vertical line of symmetry. The experimental setup and physical test bench are depicted in FIGS. 6B and 6C, respectively. At P1, the signal generator sends signals through an op amp (Analog Devices ADA4937) with auxiliary resistors which set the input impedance to 50Ω and the output signals identical to the input signal, satisfying the following equation:

$$\frac{{R}_{T} {R}_{F}}{( {R}_{T} + {P}_{S} ) ( {R}_{G} + ( {R}_{T} ‖ {R}_{S} ) )} = 1. \qquad ( 9 )$$

This equation holds when R_{T}=61.9Ω, R_{S}=50Ω, R_{F}=414Ω, and R_{G}=200Ω. The op amp converts Vᵢₙ from single-ended to differential, and the differential signals enter the AQC. At P2, a DUT of the form Z_{L}=R+jX is realized by series R_{L} and C_{L} discrete components. The 330-nH inductors (Zᵣ) resonate with corresponding capacitors (C_{L}), and R_{L} is tested at 50, 150, and 250Ω. At P3, data are converted by the oscilloscope to the frequency domain, where the logarithmic signal magnitude at the frequency of interest was observed and recorded as R_{V} varies. Measurements were made at room temperature.

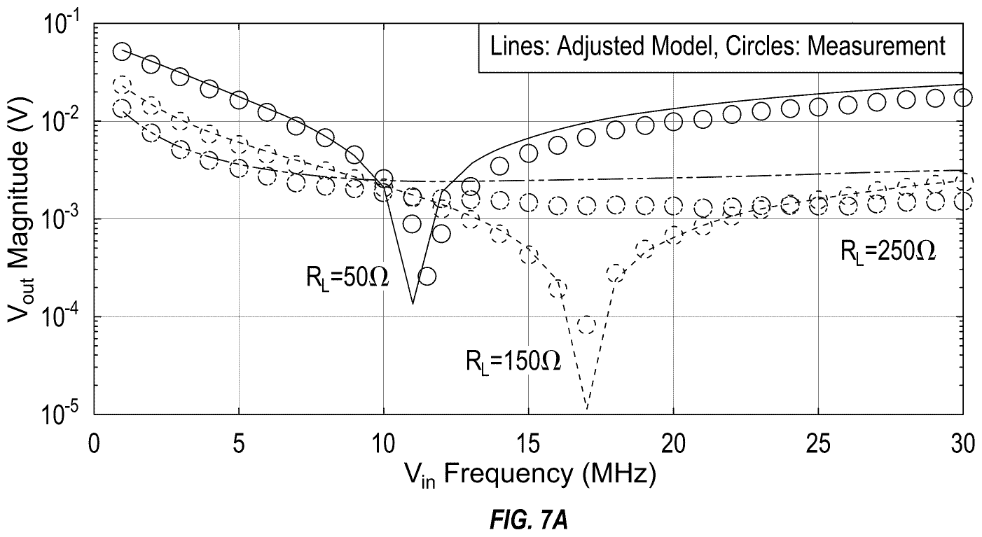

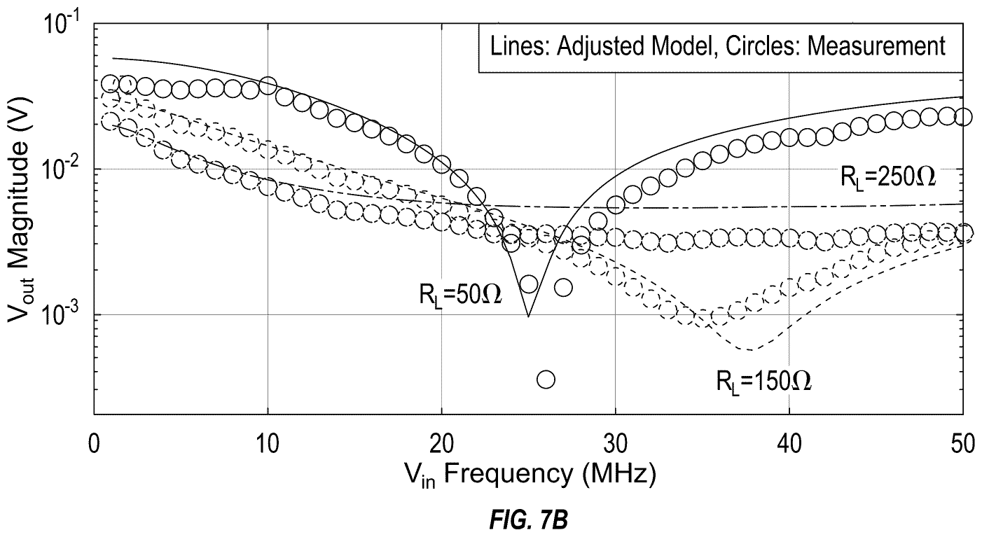

FIGS. 7A and 7B show Vₒᵤₜ magnitude versus Vᵢₙ frequency. The magnitude exhibits a null at resonance (measured to the nearest 1-MHz step). This is expected because, at resonance, the load impedance becomes purely real, matching with the real R_{V} at P1. The frequency null, which corresponds to the minimum magnitude, differs for each R_{L} value.

In FIG. 7A, C_{L} and Zᵣ are set to resonate at 10 MHZ, but the actual frequency null may vary due to parasitics. This variation can be explained in the context of FIG. 3: The contribution of the upper path to the P3 output is ideally constant across frequency. However, with a parasitic capacitor in parallel with R_{V}, the magnitude contribution of the upper path varies with frequency. This variation increases with R_{V}. The contribution of the lower path varies significantly across frequency when R_{L} is small, thereby defining the resonant frequency. The variation of the lower path decreases as R_{L} increases, resulting in a larger percentage contribution of the upper path's variation to the P3 output. The parasitics thus cause an increasing shift in the AQC's resonant frequency with increasing R_{L}.

This effect can be modeled with parasitic capacitances representing the ground plane surrounding and the close proximity of the PCB components at P2. In the model, parasitic capacitors are added to ground and in series with each P2 component, in addition to the parasitic capacitor added in parallel with R_{L}. The values of these capacitors are chosen to match the trend of measured Vₒᵤₜ magnitude v. frequency. The transconductances of M₃ and M₅ are then adjusted until the model magnitudes line up with 0.0% error in X_{C} at the calibration point (R_{L}=50Ω at 10 MHz; see Table I, which is described below).

This model adjustment to account for parasitics and chip layout is similar in concept to VNA calibration. With parasitics modeled and transconductances calibrated, the measurement and model display frequency nulls within 0.5 MHz of each other (aside from R_{L}=250Ω at 10 MHz resonance; reasons for decreased accuracy with increasing R_{L} are described later), as well as matching trends in Vₒᵤₜ magnitude across frequency for each R_{L} value.

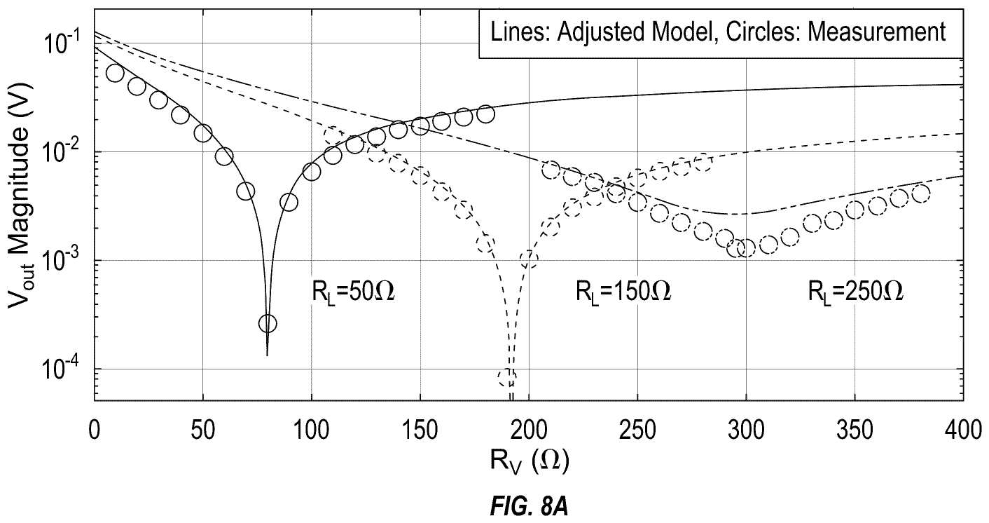

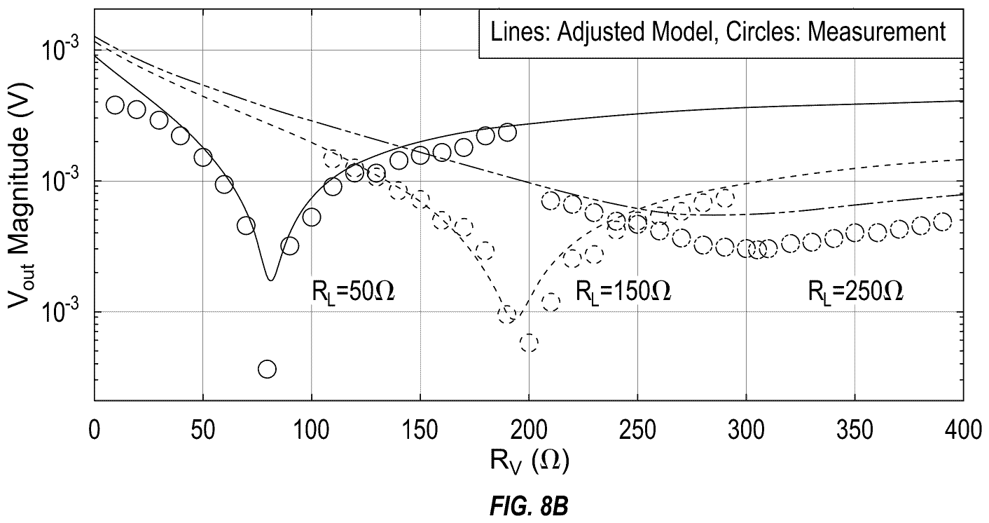

FIGS. 8A and 8B show Vₒᵤₜ magnitude versus R_{V} for different R_{L} values at the frequency nulls found in the previous plots (grouped as “10 MHz resonance” and “25 MHZ resonance” based on the frequencies at which C_{L} and Zᵣ resonate). As expected, Vₒᵤₜ magnitude is large when the gain mismatch between the aforementioned upper and lower paths is large and falls to a minimum when the path gains are matched (i.e., Gₘ₁=−Gₘ₂; see FIGS. 2 and 3).

FIG. 7B shows similar results for the 25 MHz setting, with measurement and model frequency nulls all within 3 MHz of each other. These parasitic effects could be mitigated by minimizing parasitics in the PCB design, especially by removing the ground plane immediately around and increasing the separation between the PCB components at P2.

To measure impedance which changes over time, frequency may be swept to find the frequency null, and then R_{V} would be swept at that frequency to find the match point null. Frequency and R_{V} may be iteratively swept to settle on the minimum null setting, and then data may be collected for a period of time at these ideal frequency and R_{V} values. These values may be swept at regular intervals and adjusted as needed to ensure ongoing data collection at the null. To extract the reactance of the DUT, the frequency that produces a null or minimum magnitude (fᵣₑₛ) may be placed into the resonance equation (fᵣₑₛ=1/(2π∞LC)) along with the known inductance value (L), and C may be calculated. Using C, reactance may be found: X_{C}=−1/(2πfᵣₑₛC). To extract the resistance of the DUT, the value of R_{V} that produces a minimum magnitude or null is placed into the transfer function along with known small-signal device parameters (r₀, gₘ, g_{mb}, and R_{D}), and R_{L} is calculated.

This extraction is performed, and the resulting DUT values are reported in Table I below. While fᵣₑₛ is ideally 10 or 25 MHz and R_{L} is ideally 50, 150, or 250Ω, parasitics affect the actual resistance and reactance values. Thus, the measured values are compared with the actual values including parasitics. The reactance error is within 3% for R_{L}=50Ω and 150Ω, as well as the resistance error for fᵣₑₛ=10 MHz.

The increase in error with increasing fᵣₑₛ may be due to inaccuracies in the assumptions of parasitic values, simply because inaccuracies manifest themselves increasingly with frequency. The increase in error with increasing RL is likely due to the decreased sensitivity illustrated in FIGS. 7 and 8. The measurement sensitivity is highest for small R_{L}. As R_{L} increases, the sensitivity decreases, both because of the decrease in AQC gain (which decreases the maximum magnitude) and the increasing effect of parasitics associated with R_{L} (which increases the minimum magnitude). Where the minima are less pronounced, they are difficult to measure accurately. High sensitivity, in contrast, produces distinct magnitude nulls, leading to high DUT extraction accuracy.

TABLE I
Measured DUT Values (Ω)

| | Ideal Fᵣₑₛ | | | |
|---|---|---|---|---|
| | 10 MHz | | 25 MHz | |
| | Ideal R_{L} | | | |
| | 50 | 150 | 50 | 150 |
| Actual X_{C} with parasitics | −22.8 | −34.2 | −49.0 | −70.8 |
| Measured X_{C} | −22.8 | −35.2 | −53.9 | −72.6 |
| % X_{C} Error | 0.0^{a} | 2.92 | 0.1 | 2.54 |
| Actual R_{L} with parasitics | 53.1 | 149.0 | 50.0 | 132.9 |
| Measured R_{L} | 53.9 | 152.8 | 53.9 | 152.4 |
| % R_{L} Error | 1.51 | 2.55 | 7.8 | 14.67 |

^{a}Calibration point

Table II, which is presented below, shows the performance summary and comparison to other works. Works [6], [10], [22], [23], measure impedance, but their ranges mainly cover the kHz frequencies, with the highest measuring to 2.2 MHz. Works [25], [26] measure in the same frequency range as this work, but they require choppers to achieve MHz frequencies, while this work achieves these frequencies chopper-free. [27] also uses a chopper and attenuates chopper kickback with a preceding broadband amplifier. Works [30] and [31] are included for completeness. They operate at much higher frequencies than the proposed circuit, but they are used for R_{F} communications instead of impedance measurement. The chip area, at 0.012 mm², is small but not directly comparable because the other area measurements include additional components such as excitation source, analog-to-digital converters, mixers, pads, etc. Sensitivity is calculated as the maximum step size in output magnitude close to the null. Only the Wheatstone bridge readout IC [37] shows similar sensitivity, but it does so at low frequencies only.

Isolation between P1 and P3 is reported for comparison with other AQC ICs. The isolation, calculated as the difference between the input signal magnitude and the minimum output signal magnitude for a given R_{L} value, is comparable to other AQC ICs. While the power consumption is also comparable, a target application of sub-1-Hz changes in living tissue impedance allows the IC to be powered down most of time. This makes the power figure practical for wearables. This application also allows for narrowband measurements, thus making the output noise small. This work demonstrates impedance measurement frequencies exceeding 10 MHz from a chopper-free integrated circuit, as well as high sensitivity and good isolation, making it suitable for wearable applications which require measurement frequencies in the MHz range.

TABLE II
Performance Summary and Comparison

| | | | | | | | Core | Isolation | | | |
|---|---|---|---|---|---|---|---|---|---|---|---|
| | | | | | Tech. | Frequency | Area | (dB) | Sensitivity | Power | |
| | Architecture. | IC? | Chopper? | Application | (μm) | (MHz) | (mm²) | best/worst | (μV/Ω) | (mW) | Noise |
| This | AQC | Yes | No | Impedance | 0.18 | 10-25 | 0.012 | 54/30^{a} | 406 | 25.2 | 269.3 mΩᵣₘₛ^{a} |
| Work | | | | | | | | 41/23^{b} | | | 387.7 mΩᵣₘₛ^{b} |
| | | | | | | | | | | | (Input Referred) |
| [6] | 4-Point | Yes | Yes | Impedance | 0.15 | 0.002-2 | 1.49^{c} | — | — | 0.297 | 1.63 mVᵣₘₛ |
| | | | | | | | | | | | (Output)^{d} |
| [10] | 2-Point | No | No | Impedance | — | 0.001-2.2 | — | — | — | — | — |
| [22] | 4-Point | Yes | No | Impedance | 0.35 | 0.01-0.1 | 0.91 | — | 5.1 | 0.528 | — |
| [23] | 4-Point | No | No | Impedance | — | 0.0003-0.01 | — | — | 0.285 | — | — |
| [37] | Wheatstone | Yes | No | Impedance | 0.18 | 0.000001-0.000840 | 8.6 | — | 593 | 1.5 | 24.88 nV/\sqrt{H z} |
| | Bridge | | | | | | | | | | (Input Referred) |
| [25] | 2-Point | Yes | Yes | Capacitance | 0.35 | 0.01-150 | 1.6 | — | — | 112.5 | — |
| [26] | 4-Point | Yes | Yes | Impedance | 0.18 | 0.0001-10 | 1.95 | — | — | 0.736 | 1.45 μVᵣₘₛ |
| | | | | | | | | | | | (Input Referred) |
| [27] | 2-Point | Yes | Yes | Permittivity | 0.35 | 10-3000 | 9^{e} | — | — | 4-9 | 6.7-17.8 dB |
| | | | | | | | | | | | (Noise Figure) |
| [30] | AQC | Yes | No | Comms. | 0.18 | 1500-2700 | 0.25^{e} | 37/26 | — | 86 | 10.21-10.63 dB |
| | | | | | | | | | | | (Noise Figure) |
| [31] | AQC | Yes | No | Comms. | 0.18 | 1000-7000 | 0.567^{e} | 50/36 | — | 25.2 | 16-20 dB |
| | | | | | | | | | | | (Noise Figure) |

^{a}@10 MHz;
^{b}@25 MHz;
^{c}SOC;
^{d}Single-ended;
^{e}Including pads

In preferred embodiments and implementations, the techniques utilizing an active quasi-circulator (AQC) for impedance measurement described herein benefit from specific port impedance, real-time tunability, and CMOS integration, respectively overcoming one or more of the impedance measurement challenges of frequency, sensitivity, and size. In contrast to the high impedance typically presented to a DUT (which limits measurements to the kHz range), preferred embodiments and implementations of the proposed AQC present specific port impedances which enable the measurement of DUT impedance at MHz excitation frequencies. Utilizing the AQC, a sensitive DUT measurement technique translates signals of interest to near-zero volts through resonance and matching. The translation leads to nulls in the magnitude outputs across resonance and matching sweeps, and these nulls may be used to extract complex DUT impedance using small-signal circuit analysis.

The exemplary AQC used during testing was fabricated in a 180-nm CMOS process and occupies an active area of 0.012 mm². Silicon results demonstrated complex impedance measurement at excitation frequencies of 10 MHz and 25 MHz. The proposed circuit may be suitable for wearable applications to measure living tissue characteristics, such as blood glucose, which exhibit interesting bioimpedance response in the MHz range.

### Example 2

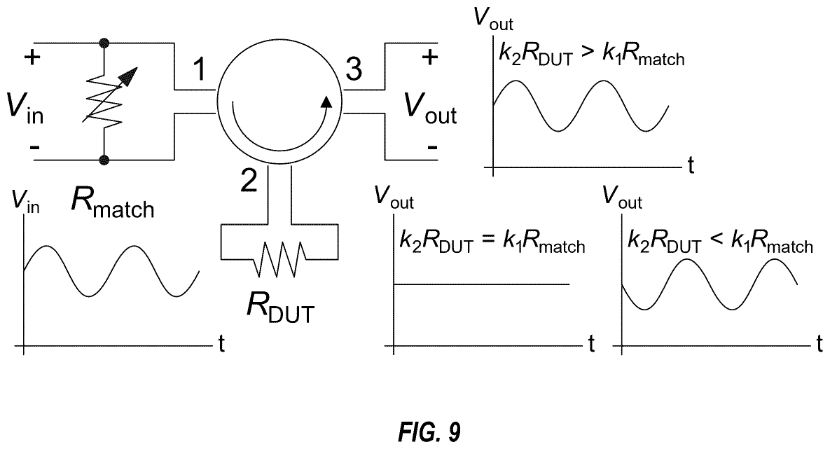

In this working example, we exploit the properties of the quasi-circulator to measure bioimpedance by placing a known load (R_{match}) at port 1 and the DUT (R_{DUT}) at port 2, as shown in FIG. 9. Then, at port 3, we observe the output signal using the following equation:

$${V}_{out} = {V}_{i n} α ( \frac{1}{1 + {k}_{1} {R}_{match}} - \frac{1}{1 + {k}_{2} {Z}_{DUT}} ) ,$$

where Vᵢₙ is the voltage applied at port 1; coefficients α, k₁, and k₂ are parameters internal to the quasi-circulator; and Z_{DUT} is the impedance of the DUT (which reduces to R_{DUT} for a purely real load). From the above equation, we can calculate the value of Z_{DUT} when the values of the other variables are known.

In the case where Z_{DUT} is a purely real load (R_{DUT}), the phase of Vₒᵤₜ relative to Vᵢₙ will be either 0° or 180°. If K₂R_{DUT}<K₁R_{match}, then Vₒᵤₜ∝−Vᵢₙ (i.e., Vₒᵤₜ=180°). If K₂R_{DUT}>K₁R_{match}, then Vₒᵤₜ∝Vᵢₙ (i.e., ∠Vₒᵤₜ=0°). This behavior is illustrated in FIG. 9.

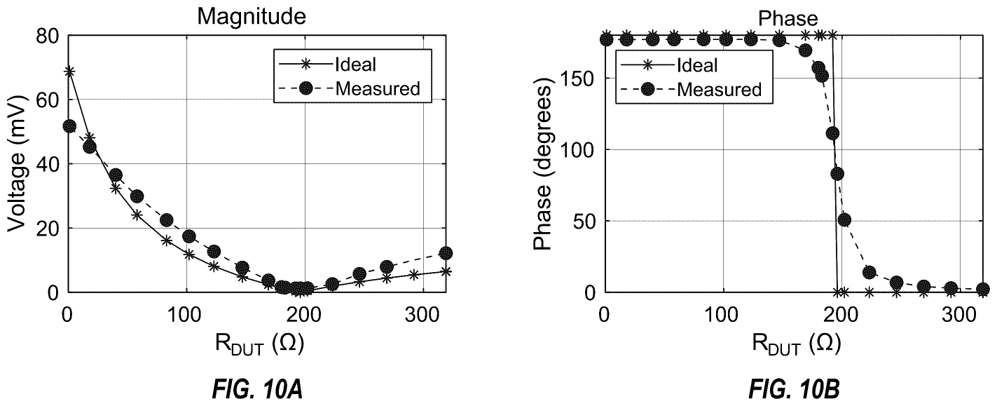

In the special case where k₂R_{DUT}=K₁R_{match}, Vₒᵤₜ=0. This is referred to as the match point of the quasi-circulator. By changing R_{match}, the match point of the quasi-circulator can be adjusted. This allows for tuning the quasi-circulator to the point where the output voltage is most sensitive to small resistive changes in the load at port 2. R_{match} was implemented as a potentiometer network on the PCB at the input of the quasi-circulator, thereby providing the ability to adjust the match point as needed. FIGS. 10A and 10B show how the magnitude and phase of the port 3 signal change with varying values of R_{DUT}. For this case, with R_{match} set to 130Ω, the match point occurs when R_{DUT}=200Ω.

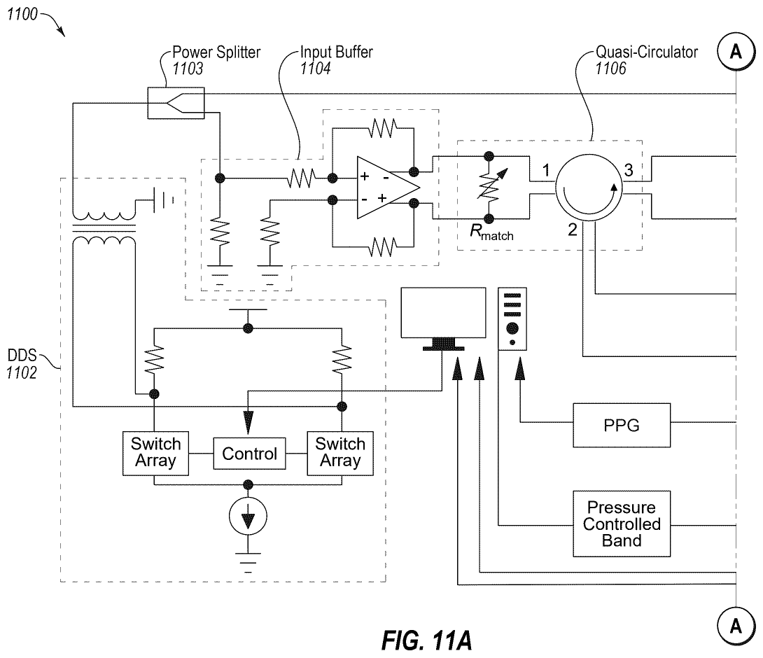

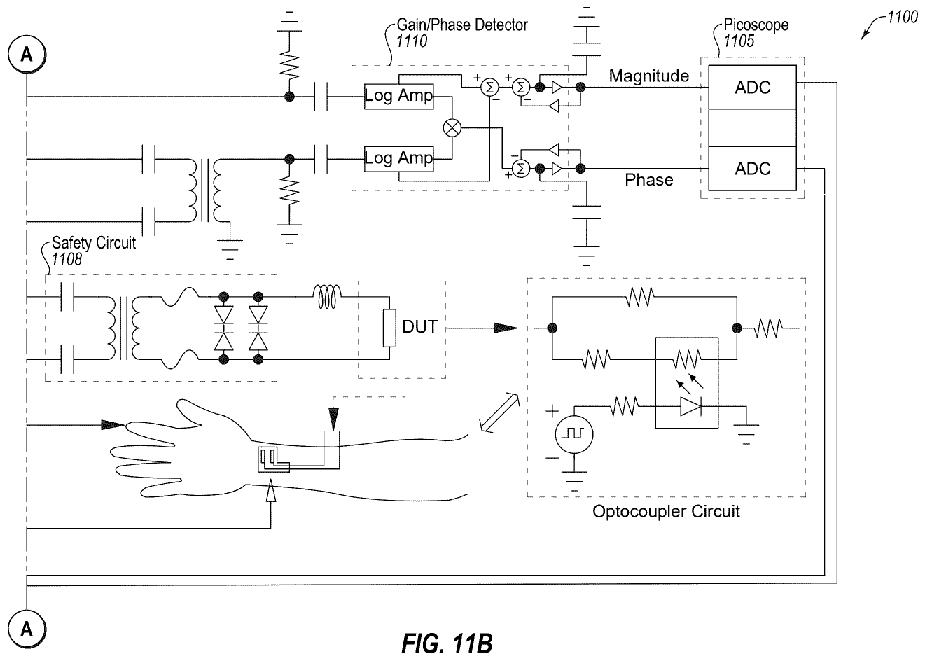

The exemplary system 1100 used in this example to measure bioimpedance consists of several main parts, as shown in FIGS. 11A and 11B. The system starts with a sinusoidal signal generated by a direct digital synthesizer (DDS) 1102, which in the example comprises an Analog Devices AD9914 DDS. The signal is sent over an SMA coaxial cable to a PCB where it is split with a splitter 1103, such as a wideband power splitter, Mini-Circuits SCP-2-1A+. The power splitter allows for maintaining impedance matching for the transmission line and ensures that equal powers of signal are sent to both the reference of the gain/phase detector and to port 1 of the quasi-circulator 1106.

An input buffer 1104, such as Analog Devices ADA4937-1 differential amplifier, is placed before port 1 of the quasi-circulator 1106 to present the proper input impedance for the power splitter 1103 and to balance the signal for the differential input to the quasi-circulator 1106.

In the example, and in preferred embodiments and implementations, the circulator is an active quasi-circulator 1106. The IC may be fabricated using CMOS technology and soldered to the system PCB. At the input to the quasi-circulator 1106 is a resistor, R_{match}, that is used to tune the system to increase sensitivity for different ranges of R_{DUT}.

In alternative embodiments and implementations, this tuning, however, may be implemented by use of digital control elements rather than physical, external resistors. For example, in some embodiments and implementations, tuning may be performed using a potentiometer, a digital potentiometer, or a digital-to-analog converter. The digital-to-analog converter may, in some cases, be implemented as a resistive digital-to-analog converter or some other digital-to-analog converter. This may provide for the ability to adjust the resistance of R_{match} dynamically through software or firmware control. These software or firmware controls may be implemented by programming bits of a shift register in an integrated circuit to switch sections of the digital-to-analog converter into or out of a circuit. For example, a resistive digital-to-analog converter may be implemented as parallel resistors, the more of which are switched into the circuit, the lower the resistance of the digital-to-analog converter due to the parallel structure. Such software or firmware controls should be considered examples of “tuning modules,” as used herein.

System 1100 further comprises a safety circuit 1108 that is placed between port 2 of the circulator 1106 and a human subject/user. This safety circuit 1108 preferably provides both DC isolation and protects against overvoltage. Safety circuit 1108 further preferably ensures that system 1100 complies with the relevant medical device safety standards, such as the standards found in IEC 60601-1.

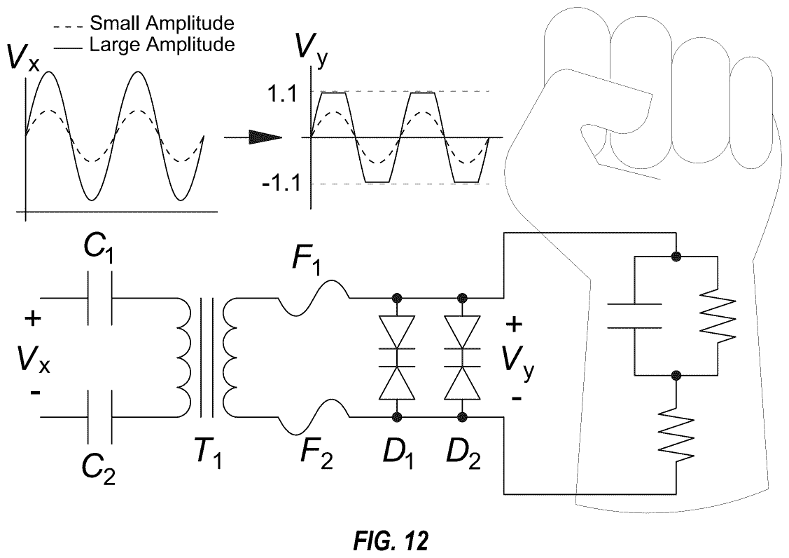

In order to provide DC isolation, two methods of safety may be implemented. First, AC coupling capacitors, C₁ and C₂, may be placed at each output of port 2 of circulator 1106, as shown in FIG. 12. In the example, a value of 10 μF was used in order to block DC and low frequency signals while providing a low impedance path for the higher frequency signals of interest. Second, a transformer with a wideband response and a winding ratio of 1:1, T₁, may be placed after the AC coupling capacitors. The combination of these two elements provides DC isolation even in the case of any single fault.

The quasi-circulator may be hardware configured to allow an absolute maximum of 1.4 mAᵣₘₛ current to be injected into the DUT, which can be either a test fixture or a human subject, preferably through a safety circuit, at port 2. This configuration ensures that the circuit is well within the safe current limits during normal operation and protects against unintentionally high input amplitudes.

Additional components may be included to provide additional protection. For example, on the secondary side of the transformer, a pair of TVS diodes, D₁ and D₂, may be placed in parallel with the DUT. These diodes may be designed to clip at 1.1 Vₚₖ if overvoltage is presented, thus protecting the body from excessive current.

Fuses F₁ and F₂ may be designed to open in the event that the TVS diodes sink excessive current over an extended period of time.

Once the signal has passed through the body, it is output at port 3 of the quasi-circulator. At the output of port 3, another transformer may be placed. This transformer acts as a balun to convert the differential quasi-circulator output to a single-ended signal. This single-ended signal is then input into a gain and phase detector 1110. In the example, the detector was a chip, namely, the Analog Devices AD8302 chip. The AD8302 chip is a gain and phase detector designed to compute the relative magnitude and phase between two signals. Both the magnitude and phase signals may be output as DC voltages between 0-1.8 V. The quasi-circulator port 3 signal may then be compared to a reference signal from the power splitter. The output voltage from the AD8302, representing the magnitude of the received signal, is given by the following equation:

$${V}_{MAG} = {R}_{F} {I}_{SLP} \cdot log ( \frac{{V}_{INA}}{{V}_{INB}} ) + {V}_{CP} ,$$

where R_{F}I_{SLP}=0.6 V/decade and V_{CP}=0.9 V. For the experimental setup, VINA was the reference signal and V_{INB} was the signal from port 3 of the quasi-circulator. By solving for V_{INB}, we could calculate the output voltage from port 3 of the quasi-circulator as:

$${V}_{INB} = \frac{{V}_{INA}}{{10}^{{V}_{MAG} - {V}_{CP} / {R}_{F} {I}_{SLP}}}$$

From these equations, we can see that a maximum V_{MAG} voltage is presented when the quasi-circulator output voltage, V_{INB}, is at a minimum, thereby demonstrating the highest sensitivity to impedance changes when the quasi-circulator output voltage is small. The voltage output from the AD8302 for the phase difference between the two signals is given by:

$${V}_{PHS} = - {R}_{F} {I}_{Φ} ( Φ ( {V}_{INA} ) - Φ ( {V}_{INB} ) - 90 ° ) + {V}_{CP} ,$$

where R_{F}I_{Φ}=10 mV/degree. Solving for phase difference between the two signals yields:

$$Φ ( {V}_{INA} ) - Φ ( {V}_{INB} ) = \frac{{V}_{PHS} - {V}_{CP}}{- {R}_{F} {I}_{Φ}} + 90 °$$

To detect small impedance changes, we want to operate in a region where we have the highest sensitivity to change. Thus, we want to maximize the derivative of Vₒᵤₜ v. R_{DUT}. While FIG. 10A shows a high derivative at the point where R_{DUT} approaches 0Ω, a higher derivative can be found by dividing a reference (e.g., V_{ref}) by Vₒᵤₜ. With this division performed, the derivative is highest near the match point. The gain/phase detector in our system performs this division in the magnitude calculation, thereby producing the highest sensitivity near the match point. The phase calculation shows a linear relationship between the phase in voltage and the phase in degrees, so the sensitivity of the system is the same as the sensitivity of Vₒᵤₜ in FIG. 10B, both of which exhibit a high derivative when R_{DUT} is at the match point.

The AD8302 has 2.2 μF capacitors placed at its input. These capacitors set the time constants for the output signals to 7.26 ms. Because we are interested in pulsatile signals on the order of 1-3 Hz, a 7.26 ms time constant provides enough averaging while still being able to respond to the signals of interest. In the example, the V_{MAG} and V_{PHS} pins were then sampled by a Picoscope (see 1105 in FIG. 11B) 5444D MSO at a rate of 1 ksps.

In order to confirm that variations in the bioimpedance signal are due to the heartbeat, a photoplethysmography (PPG) sensor was integrated into the system. In the example, the MAX30101 PPG sensor was used to track the heartbeat of the subject. The sensor was placed on the index finger of the same arm from which the bioimpedance data was taken.

In order to keep the pressure applied to the electrodes constant, a pressure-controlled band was used in the example. The band has 4 force sensors positioned on a platform on top of the electrodes to determine the applied pressure. A servomotor was used to raise or lower the platform and adjust the pressure. The target pressure can be programmed to the device, and the servomotor was activated until the force sensors measure the desired pressure. The device was strapped to the user's arm with a hook and loop watch strap and controlled by an Arduino Uno.

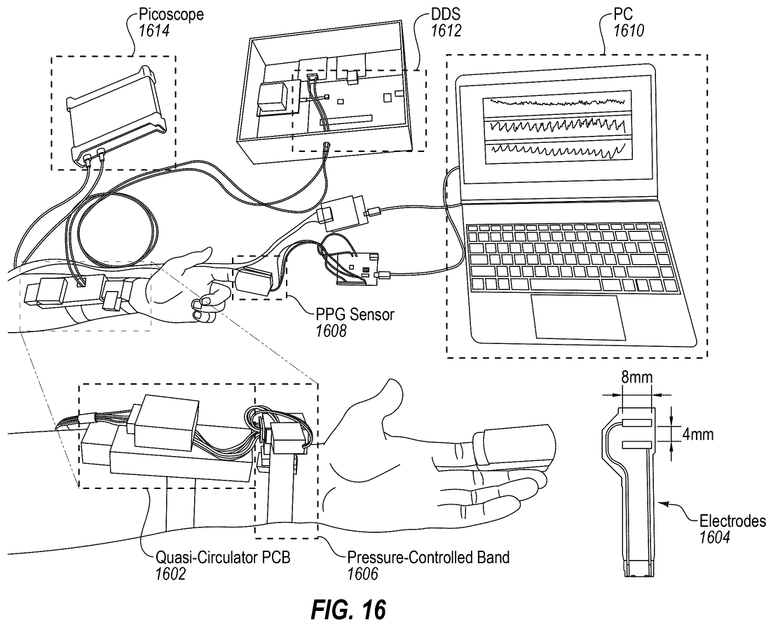

In the example, the entire system was controlled with a Python script running on a PC 1610 (see FIG. 16). This allowed for setting the frequency and amplitude output of the DDS 1612 and collection of data from the Picoscope 1614 and PPG sensor 1608 simultaneously.

To evaluate the functionality of the system, we connected a test fixture to port 2 of the quasi-circulator using a potentiometer soldered to the system test board. The potentiometer was set at various values between 0Ω and 435Ω. The resulting magnitude and phase outputs are plotted in FIGS. 10A and 10B for various load values at 9 MHZ and an input amplitude of 156 mVᵣₘₛ, with R_{match} set to 130Ω.

In FIG. 10A, the magnitude of the measured port 3 signal behaves as anticipated: When the resistance of the load is much lower than the match, e.g., 0Ω, the resulting magnitude is large. As the load resistance approaches the match point, the magnitude approaches a minimum. Once the load resistance passes the match point, the magnitude increases. With R_{match} set to 130Ω, the match point is measured to be about 200Ω.

FIG. 10B also shows the rapid phase change that occurs around the match point. When the load resistance is smaller than the match point, the output phase is 180°. When the load resistance is larger than the match point, the output phase is 0°. Close to the match point, the phase experiences a rapid phase shift between 180° and 0°.

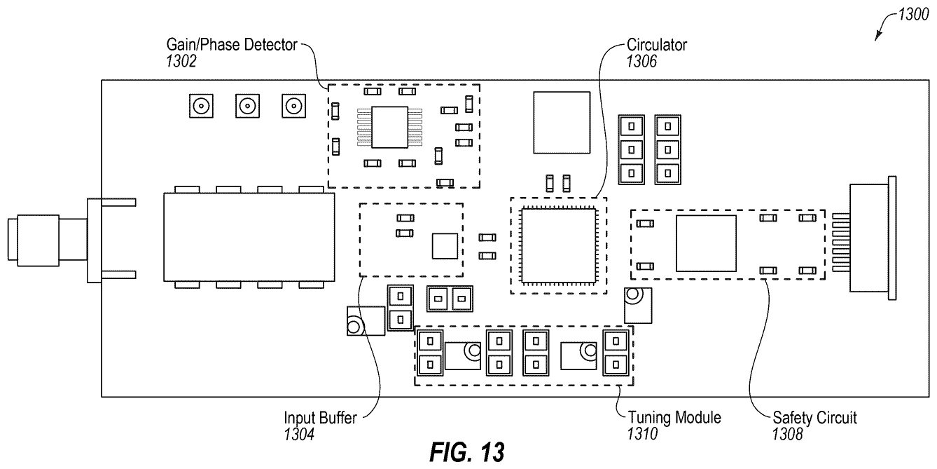

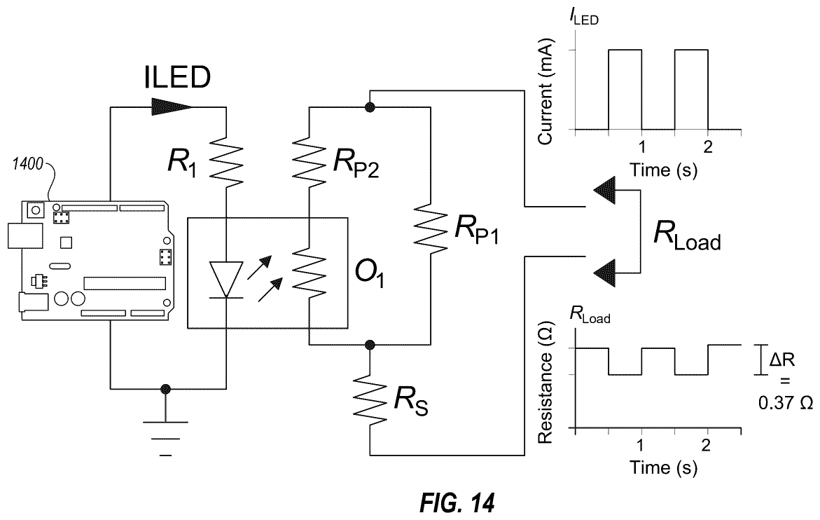

FIG. 13 illustrates an example of a PCB 1300 that may be used in some quasi-circulator systems described herein. PCB 1300 comprises a gain/phase detector 1302, an input buffer 1304, a circulator 1306, and a safety circuit 1308. An impedance varying component or tuning module 1310 is also shown, which may be used to vary R_{match}.

To determine which region of operation would be most sensitive to small impedance changes, a time-varying resistance circuit was designed and built. This circuit is shown in FIGS. 11A and 11B. Component O₁ is an optocoupler consisting of an LED and a photocell. When no current passes through the LED, the resistance of the photocell, R_{O1}, is very large (>25 MΩ, considered as an open). When current passes through the LED, the resistance drops proportional to the amount of current through the LED. By pulsing the LED current on and off, the overall change in resistance, ΔR, is given by:

$$Δ R = {R}_{P 1} - \frac{{R}_{P 1} ( {R}_{P 2} + {R}_{O 1} )}{{R}_{P 1} + ( {R}_{P 2} + {R}_{O 1} )}$$

By selecting (R_{P2}+R_{O1})>>R_{P1}, ΔR can be made extremely small. For the example tests, the following values were selected: R_{S}=226Ω, R_{P1}=27Ω, and R_{P2}+R_{O1}=1.96 kΩ (when the LED is on). This creates a ΔR of 0.37Ω. The optocoupler was cycled on and off at a rate of 1 Hz by means of an Arduino microcontroller. This allows the variable resistance to mimic the rate of a typical cardiac cycle. The circuit was attached to port 2 of the quasi-circulator, and R_{match} was varied from 100Ω to 200Ω.

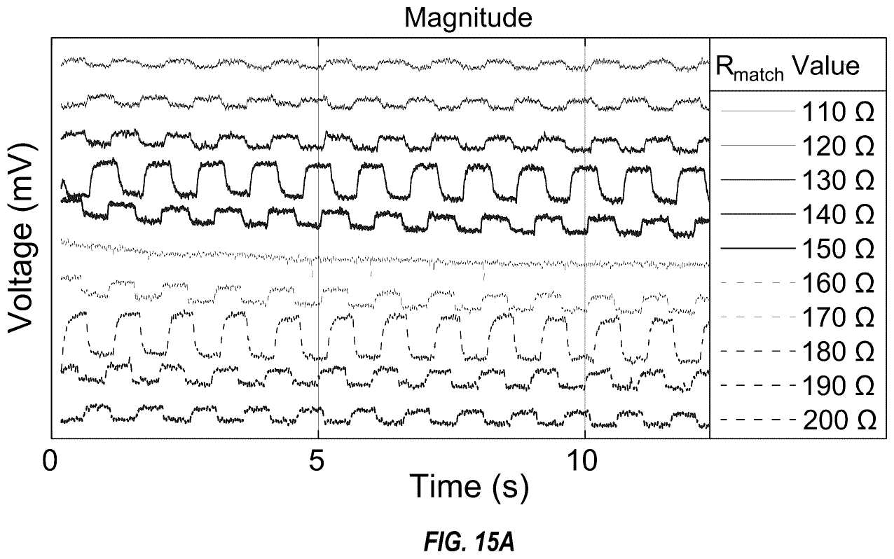

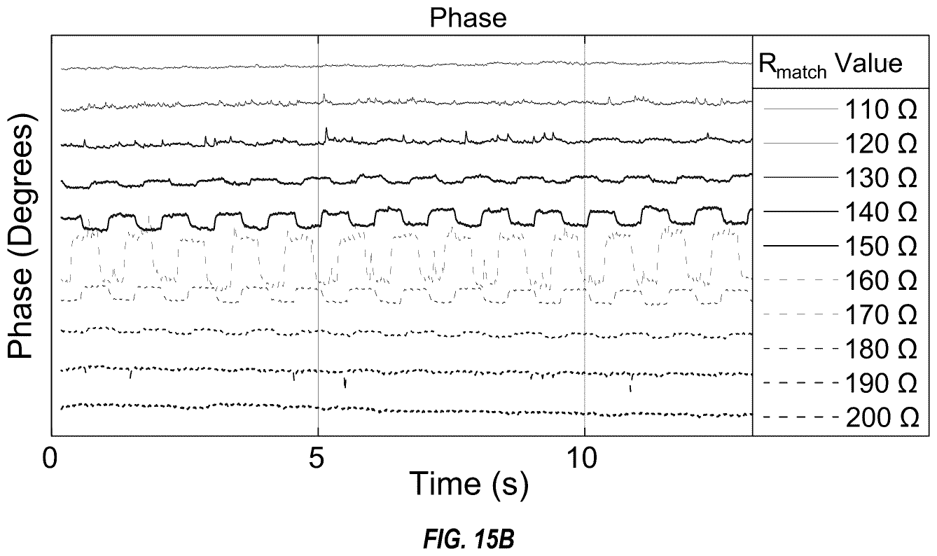

FIG. 15 shows the output magnitude and phase variations for different values of R_{match}. The relative pulse amplitudes in the magnitude and phase follow the expected behavior. When the R_{load} value is far from the match point, there is little sensitivity in the output magnitude and phase. As the match point approaches the value of R_{load}, the pulse amplitude increases. When the load is closely matched, in this case when R_{match}=160Ω, the pulse amplitude in the magnitude goes to zero. This correlates to the match point in FIG. 10A, where the slope of the magnitude goes to zero. In the phase, we see the largest pulse amplitude right at the match point. This agrees with FIG. 10B, where the phase experiences a rapid transition at the match point.

Human experiments were conducted under approval from the Brigham Young University Institutional Review Board (IRB #F2020-268). Five individuals were chosen to participate in the study. Data for each participant are shown in the following Table:

| Participant ID # | Sex | Age | BMI |
|---|---|---|---|
| 1 | Male | 22 | 18.1 |
| 2 | Male | 25 | 19.6 |
| 3 | Female | 23 | 23.6 |
| 4 | Female | 24 | 23.0 |
| 5 | Male | 28 | 27.4 |

FIG. 16 shows the setup used for human experiments. The quasi-circulator PCB 1602 was mounted inside an enclosed, 3D printed housing and mounted to the forearm of each subject with an elastic watch strap. The electrodes were fabricated on a flexible PCB and connected to a quasi-circulator system PCB 1602 by means of a Molex 52271-0769 connector. The electrodes 1604 were gold plated copper, and each electrode was a 2 mm×8 mm rectangle spaced 4 mm apart. The electrodes were placed directly on top of the radial artery, with each electrode running perpendicular to the artery. The pressure-controlled band 1606 was placed directly on top of the electrodes and strapped to the participant's wrist, and the PPG sensor 1608 was clipped onto the participant's finger. The rest of the test equipment was located on the table next to the participant and connected by means of coaxial cables. Participants were seated for the duration of the experiment with their arm resting on a table.

It has been shown that the contact impedance between electrodes and skin drifts over time before reaching an acclimation point. To reduce the time and degree of acclimation, electrode gel was applied between the electrodes and the skin of the participant at the beginning of each session. An inductor was placed in series with the body tissue to cancel the capacitive part of the body impedance so that the body impedance would match a resistive load. This inductor was used to create a resonant point with the reactive component of the body impedance. A 1 μH inductor was used to place the resonant point within our frequency range of interest. The inductor is soldered to the quasi-circulator system board in series with the electrodes.

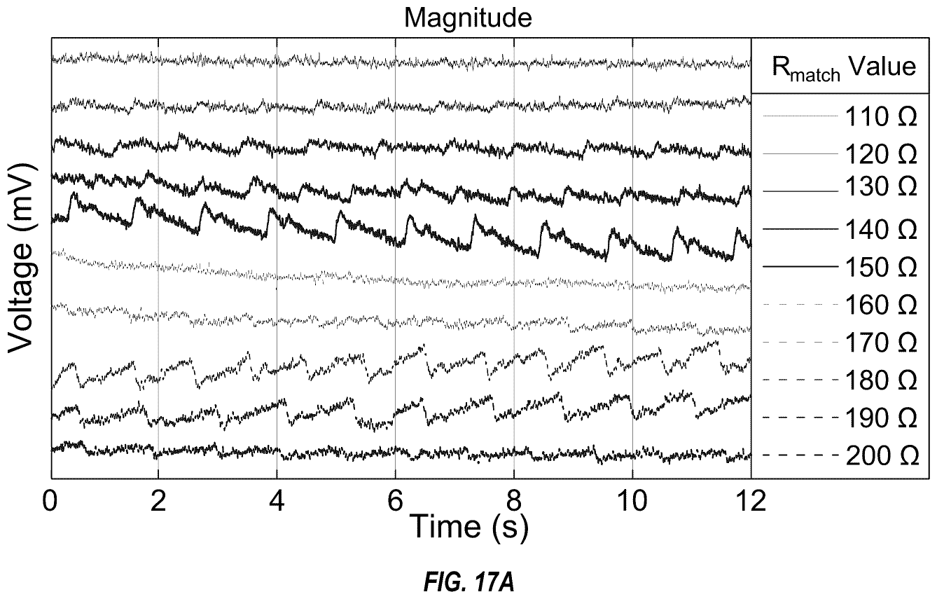

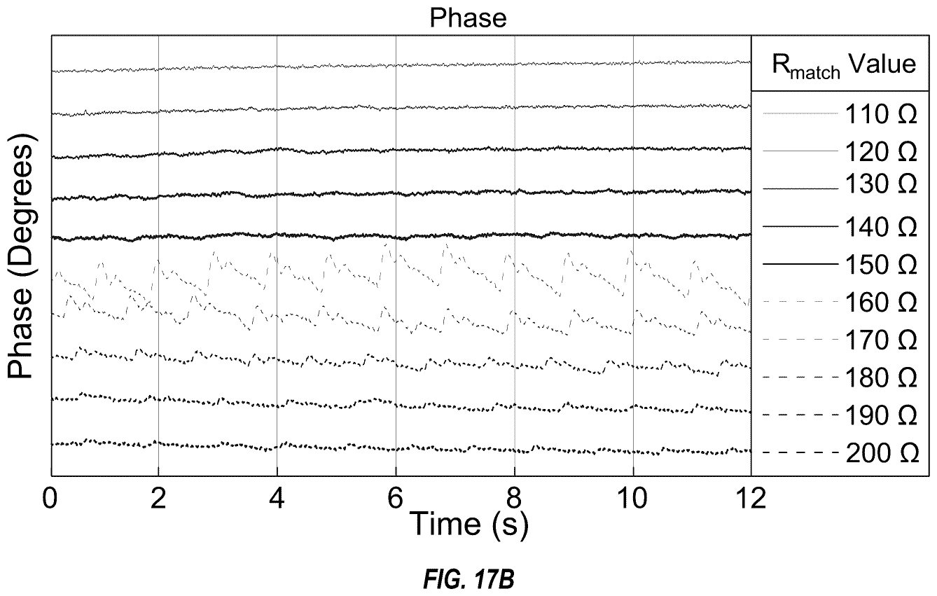

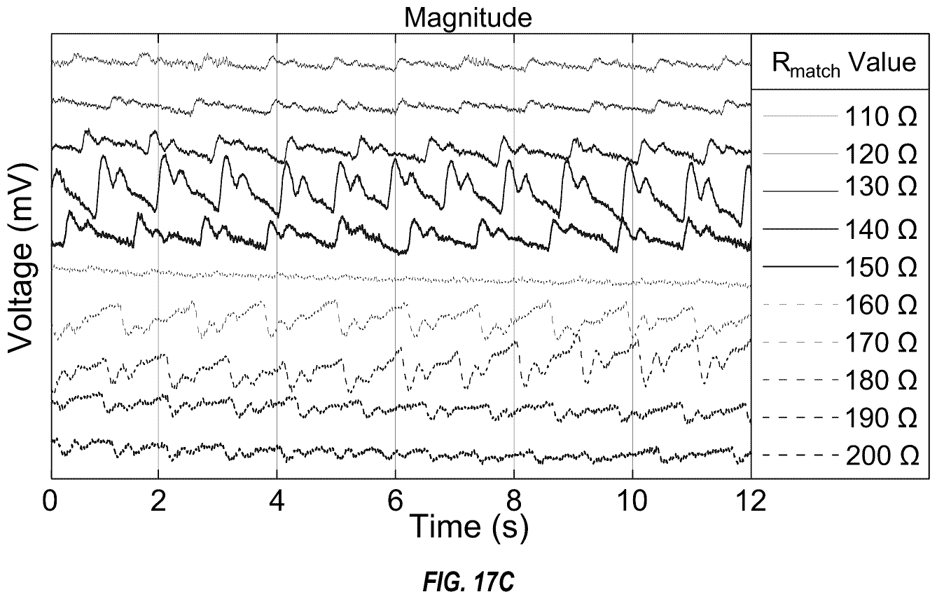

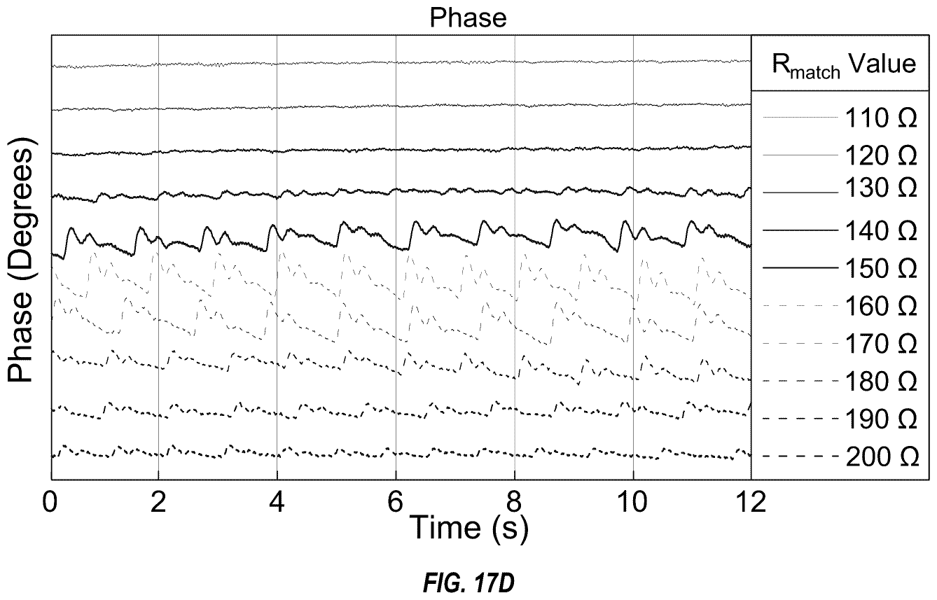

For each subject, R_{match} was initially set to a much lower value than was expected to match with the body. The input frequency was then swept in increments of 1 MHz to find the approximate resonant frequency for that individual. At the measured resonant frequency, 15-second dwells were then conducted at each R_{match} value, where R_{match} was increased in 10Ω increments from 100Ω-200Ω. FIGS. 17A and 17B show the output pulse amplitudes for participant 1. For participant 1, the input frequency was set to 9 MHZ, and 15-second dwells were conducted at each R_{match} value.

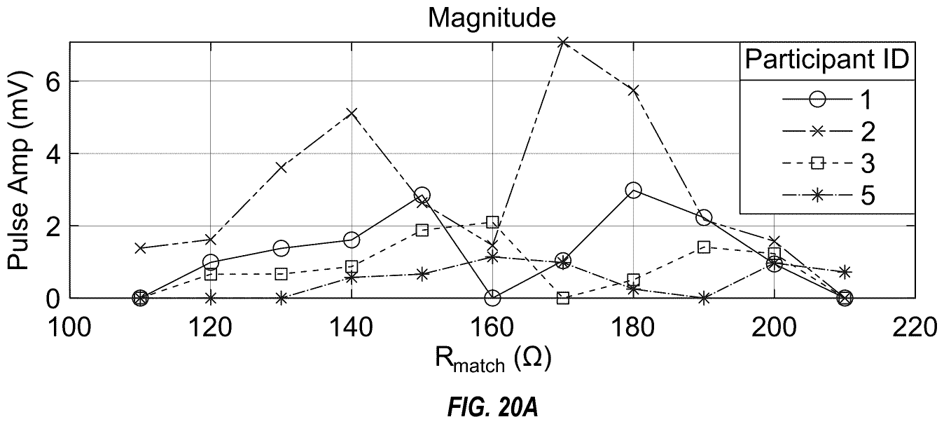

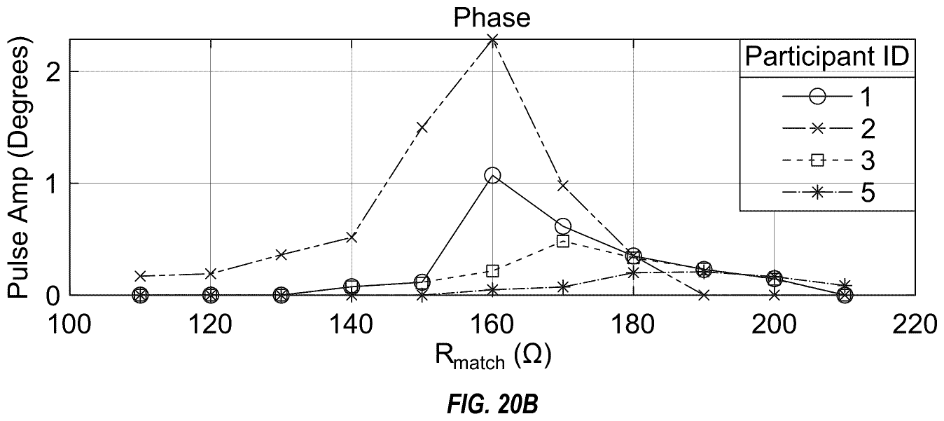

FIGS. 17C and 17D show the pulse amplitudes for participant 2. For participant 2, the input frequency was set to 11 MHZ, and the same 15-second dwells were conducted for each R_{match} value. Data for participants 1, 2, 3, and 5 are summarized in FIGS. 20A and 20B.

For participant 4, the R_{match} potentiometer setup was unable to be set at a high enough value to reach the match point. As such, the data from participant 4 has not been included. Comparing FIG. 17A with FIG. 15A, we can see similar behavior in the magnitude response. The output magnitude shows the largest response to the pulsing load with R_{match} values near the match point of 160Ω, but very little response at the match point. In contrast, the output phase in FIG. 17B shows the greatest response at the match point, mirroring the behavior shown in FIG. 15B.

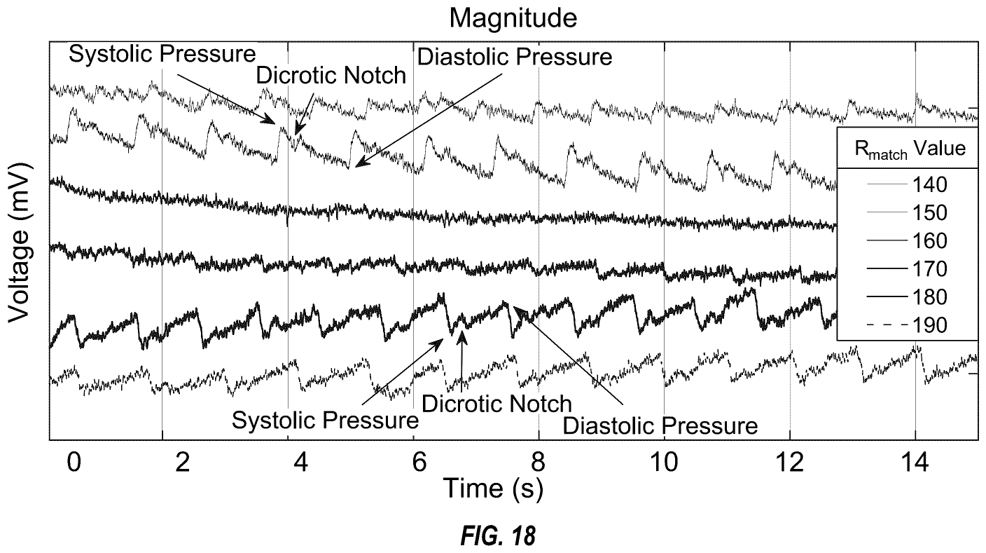

From the magnitude response of participant 1 in FIG. 18, we can gain insight by observing the locations of the systolic and diastolic pressures.

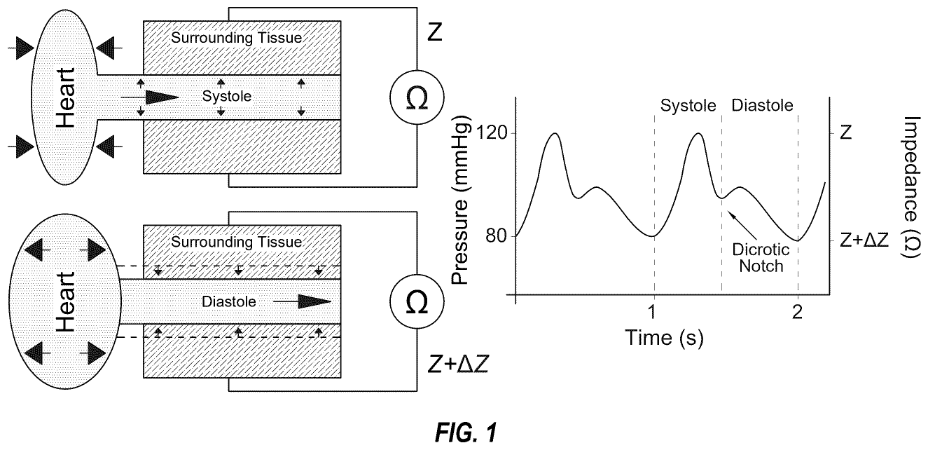

By using the dicrotic notch to delineate the systole and diastole phases, as shown in FIG. 1, we can determine how the systolic and diastolic blood volumes affect the impedance measurement. For R_{match} values lower than the match point (such as when R_{match}=150Ω) the systolic pressure corresponds to a higher magnitude output than the diastolic pressure. For R_{match} values higher than the match point (such as when R_{match}=180Ω), the systolic pressure corresponds to a lower magnitude output than the diastolic pressure. This aligns with the analysis of FIG. 1 that the systolic pressure has a lower impedance than the diastolic pressure due to a higher blood volume in the tissue.

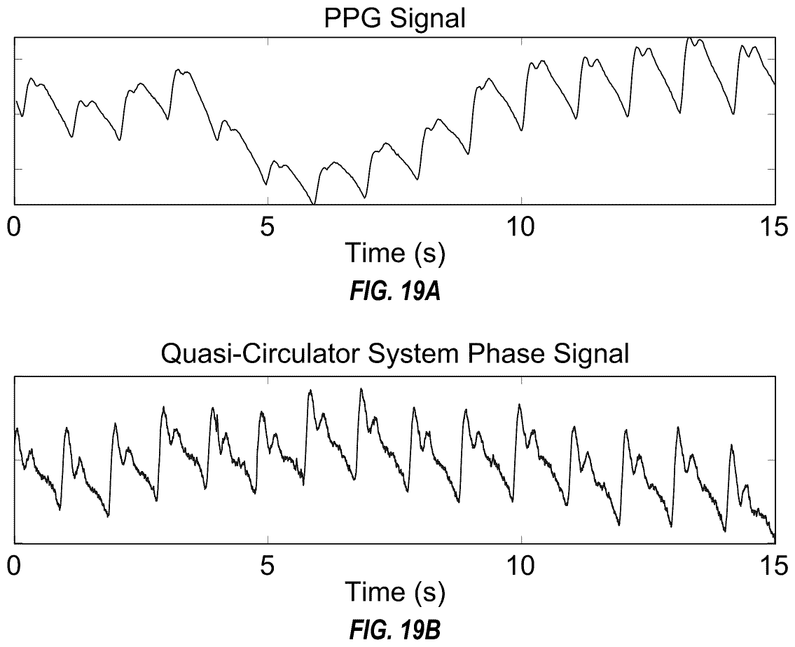

FIGS. 19A and 19B show the simultaneous PPG signal and BIP signal collected with the quasi-circulator system. The BIP data are the output phase signal from participant #1 with R_{match} set to 160Ω, which corresponds to the match point for this individual. It is of interest to note that the BIP signal is less susceptible to artifacts caused by motion or other perturbations. This was observed across all participants. Additionally, the BIP signal displays a more detailed shape with more defined features of the pulse as compared to the PPG signal.

FIGS. 20A and 20B show the pulse amplitudes for participants 1, 2, 3, and 5 at each R_{match} value. For each participant, the pulse amplitude in the magnitude is highest on either side of the match point, while the pulse amplitude in the phase is highest at the match point. For participants 1 and 2, the match point occurred when R_{match} was set to 160Ω. Participant 3 was matched when R_{match} was set to 170Ω, while an R_{match} value of 190Ω best matched participant 5.

Peak pulse amplitude also varied among participants. Participant 1 had a maximum pulse amplitude of 2.98 mV in the magnitude output and 1.07° of phase change. Participant 2 topped out with 7.08 mV of pulsatile magnitude variation and a maximum phase change of 2.29°. Participant 3 had peak pulse amplitudes of 2.10 mV and 0.48°, while participant 5 had the smallest pulse amplitudes at 1.14 mV and 0.21°.

For all participants except participant 4, an R_{match} value between 150-200Ω was sufficient to properly match the impedance of the participant, while participant 4 needed an R_{match} value larger than our maximum of 210Ω. A lower R_{match} value corresponds to a lower impedance value. The two participants with the lowest BMI, participants 1 and 2, had the lowest match impedance, and therefore the lowest tissue impedance, while the participants with higher BMIs tended to have higher impedance values, although it is a small sample size.

The two participants with lower BMIs also had larger pulse amplitudes than the participants with higher BMIs, which could be due to arterial depth, other physiological variations, or electrode placement, as precise placement over the radial artery leads to larger amplitudes.

A system for impedance plethysmography, in some cases, MHz bioimpedance plethysmography, is proposed and demonstrated herein. In some embodiments, the system incorporates a programmable signal excitation source, an integrated active quasi-circulator, a tunable match resistance, a safety circuit, a gain/phase detector, and/or data acquisition hardware. The core circuit preferably fits within the size of a wrist-worn device. The system is tested by measuring human pulsatile signals with dynamically-optimizable sensitivity, demonstrating the system's capability for MHz BIP. Because existing BIP systems are limited to kHz frequencies, this system provides new opportunities for researchers to study the response of human tissues to MHz excitations, where blood glucose, in particular, has previously shown promising correlation. In addition, measurements show excellent sensitivity to sub-1-Ω impedance changes. The system may be valuable for applications in, for example, blood glucose monitoring, arrhythmia detection, apnea monitoring, and oximetry verification. Human subject test results successfully demonstrate the ability to measure bioimpedance changes due to the cardiac cycle with superior signal quality over PPG measurements.

Moreover, the sensitivity to small impedance changes may be adjusted by tuning the match point of the quasi-circulator, which can be done by using, for example, a tuning module, which, as discussed above, may be implemented by various hardware, software, and/or firmware elements in such a wearable device. By adjusting the match point to areas of maximum sensitivity, the output response to small impedance changes can be increased substantially.

### REFERENCES

[1] Y. Wu, D. Jiang, A. Bardill, S. de Gelidi, R. Bayford, and A. Demosthenous, “A high frame rate wearable EIT system using active electrode ASICs for lung respiration and heart rate monitoring,” IEEE Transactions on Circuits and Systems I: Regular Papers, vol. 65, no. 11, pp. 3810-3820, 2018.
[2] T. R. Sulla, S. J. Talavera, C. E. Supo, and A. A. Montoya, “Non-invasive glucose monitor based on electric bioimpedance using AFE4300,” in 2019 IEEE XXVI International Conference on Electronics, Electrical Engineering and Computing (INTERCON), August 2019, pp. 1-3.
[3] J. Li, T. Igbe, Y. Liu, Z. Nie, W. Qin, L. Wang, and Y. Hao, “An approach for noninvasive blood glucose monitoring based on bioimpedance difference considering blood volume pulsation,” IEEE Access, vol. 6, pp. 51 119-51 129, 2018.
[4] D. K. Kamat, D. Bagul, and P. M. Patil, “Blood glucose measurement using bioimpedance technique,” Advances in Electronics, vol. 2014, pp. 1-5, December 2014.
[5] P. Kassanos, I. F. Triantis, and A. Demosthenous, “A CMOS magnitude/phase measurement chip for impedance spectroscopy,” IEEE Sensors Journal, vol. 13, no. 6, pp. 2229-2236, 2013.
[6] S. Rodriguez, S. Ollmar, M. Waqar, and A. Rusu, “A batteryless sensor ASIC for implantable bio-impedance applications,” IEEE Transactions on Biomedical Circuits and Systems, vol. 10, no. 3, pp. 533-544, June 2016.
[7] J. Schneider, M. Schroth, M. Holzhey, T. Blocher, and W. Stork, “An approach to improve impedance plethysmography on the wrist by using adaptive feedback control,” in 2017 IEEE Sensors Applications Symposium (SAS), 2017, pp. 1-6.
[8] D. Allegri, D. Vaca, D. Ferreira, M. Rogantini, and D. Barrettino, “Real-time monitoring of the hydration level by multi-frequency bioimpedance spectroscopy,” in 2017 IEEE International Instrumentation and Measurement Technology Conference (12MTC). Turin, Italy: IEEE, May 2017, pp. 1-6.
[9] A. Caduff, M. S. Talary, M. Mueller, F. Dewarrat, J. Klisic, M. Donath, L. Heinemann, and W. A. Stahel, “Non-invasive glucose monitoring in patients with Type 1 diabetes: A Multisensor system combining sensors for dielectric and optical characterisation of skin,” Biosensors and Bioelectronics, vol. 24, no. 9, pp. 2778-2784 May 2009.
[10] T. Addabbo, A. Fort, M. Mugnaini, L. Parri, M. Pinzi, V. Vignoli, P. K. Mvemba, M. Becatti, V. Barygina, N. Taddei, and C. Fiorillo, “On the suitability of low-cost compact instrumentation for blood impedance measurements,” IEEE Transactions on Instrumentation and Measurement, vol. 68, no. 7, pp. 2412-2424, 2019.
[11] M. Khalighi, B. Vosoughi Vahdat, M. Mortazavi, W. Hy, and M. Soleimani, “Practical design of low-cost instrumentation for industrial electrical impedance tomography (EIT),” in 2012 IEEE International Instrumentation and Measurement Technology Conference Proceedings, 2012, pp. 1259-1263.
[12] O. Elhadidy, S. Shakib, K. Krenek, S. Palermo, and K. Entesari, “A wide-band fully-integrated CMOS ring-oscillator PLL-based complex dielectric spectroscopy system,” IEEE Transactions on Circuits and Systems I: Regular Papers, vol. 62, no. 8, pp. 1940-1949, 2015.
[13] M. Bakhshiani, M. A. Suster, and P. Mohseni, “A 9 MHZ-2.4 GHz fully integrated transceiver IC for a microfluidic-CMOS platform dedicated to miniaturized dielectric spectroscopy,” IEEE Transactions on Biomedical Circuits and Systems, vol. 9, no. 6, pp. 849-861, 2015.
[14] F. G. Baptista and J. V. Filho, “A new impedance measurement system for PZT-based structural health monitoring,” IEEE Transactions on Instrumentation and Measurement, vol. 58, no. 10, pp. 3602-3608, 2009.
[15] L. E. Sebar, L. Iannucci, E. Angelini, S. Grassini, and M. Parvis, “Electrochemical impedance spectroscopy system based on a Teensy board,” IEEE Transactions on Instrumentation and Measurement, vol. 70, pp. 1-9, 2021.
[16] Analog Devices, “1 MSPS, 12-bit impedance converter, network analyzer,” 2005, AD5933 Datasheet.
[17] A. Caduff, P. Ben Ishai, and Y. Feldman, “Continuous noninvasive glucose monitoring; Water as a relevant marker of glucose uptake in vivo,” Biophysical Reviews, vol. 11, no. 6, pp. 1017-1035, 2019.
[18] A. Caduff, E. Hirt, Y. Feldman, Z. Ali, and L. Heinemann, “First human experiments with a novel non-invasive, non-optical continuous glucose monitoring system,” Biosensors and Bioelectronics, vol. 19, no. 3, pp. 209-217, November 2003.
[19] J.-Y. Shim and J.-Y. Chung, “Complex permittivity measurement of artificial tissue emulating material using open-ended coaxial probe,” IEEE Sensors Journal, vol. 20, no. 9, pp. 4688-4693, 2020.
[20] J. Kilpijarvi, J. Tolvanen, J. Juuti, N. Halonen, and J. Hannu, “A non-invasive method for hydration status measurement with a microwave sensor using skin phantoms,” IEEE Sensors Journal, vol. 20, no. 2, pp. 1095-1104, 2020.
[21] Zurich Instruments, “SHFLI 8.5 GHZ lock-in amplifier,” 2022.
[22] J. Sacristan-Riquelme, F. Segura-Quijano, A. Baldi, and M. Teresa Oses, “Low power impedance measurement integrated circuit for sensor applications,” Microelectronics Journal, vol. 40, no. 1, pp. 177-184, 2009.
[23] S. Poff, D. Tebbs, R. C. Davis, and S. W. Chiang, “Pulsatile impedance monitoring circuit,” in 2023 Intermountain Engineering, Technology and Computing (IETC), 2023, pp. 203-208.
[24] A. Manickam, A. Chevalier, M. McDermott, A. D. Ellington, and A. Hassibi, “A CMOS electrochemical impedance spectroscopy (EIS) biosensor array,” IEEE Transactions on Biomedical Circuits and Systems, vol. 4, no. 6, pp. 379-390, 2010.
[25] D. Bianchi, G. Ferrari, A. Rottigni, and M. Sampietro, “CMOS impedance analyzer for nanosamples investigation operating up to 150 MHz with sub-aF resolution,” IEEE Journal of Solid-State Circuits, vol. 49, no. 12, pp. 2748-2757, 2014.
[26] S.-I. Cheon, S.-J. Kweon, Y. Kim, J. Koo, S. Ha, and M. Je, “A polar-demodulation-based impedance-measurement IC using frequency-shift technique with low power consumption and wide frequency range,” IEEE Transactions on Biomedical Circuits and Systems, vol. 15, no. 6, pp. 1210-1220, 2021.
[27] M. Bakhshiani, M. A. Suster, and P. Mohseni, “A broadband sensor interface IC for miniaturized dielectric spectroscopy from MHz to GHz,” IEEE Journal of Solid-State Circuits, vol. 49, no. 8, pp. 1669-1681, 2014.
[28] A. Caduff, F. Dewarrat, M. Talary, G. Stalder, L. Heinemann, and Y. Feldman, “Non-invasive glucose monitoring in patients with diabetes: A novel system based on impedance spectroscopy,” Biosensors and Bioelectronics, vol. 22, no. 5, pp. 598-604, 2006.
[29] V. Valente and A. Demosthenous, “Wideband fully-programmable dual-mode CMOS analogue front-end for electrical impedance spectroscopy,” Sensors, vol. 16, no. 8, 2016.
[30] Y. Zheng and C. E. Saavedra, “Active quasi-circulator MMIC using OTAs,” IEEE Microwave and Wireless Components Letters, vol. 19, no. 4, pp. 218-220, 2009.
[31] B. Tang, X. Gui, J. Xu, Q. Xia, and L. Geng, “A dual interference-canceling active quasi-circulator achieving 36-dB isolation over 6-GHz bandwidth,” IEEE Microwave and Wireless Components Letters, vol. 29, no. 6, pp. 409-411, 2019.
[32] B. Tang and L. Geng, “A survey of active quasi-circulators,” Journal of Semiconductors, vol. 41, no. 11, 2020.
[33] D. D. Stupin, E. A. Kuzina, A. A. Abelit, A. K. Emelyanov, D. M. Nikolaev, M. N. Ryazantsev, S. V. Koniakhin, and M. V. Dubina, “Bioimpedance spectroscopy: Basics and applications,” ACS Biomaterials Science & Engineering, vol. 7, no. 6, pp. 1962-1986, 2021.
[34] Analog Devices, “Ultralow distortion differential ADC driver,” 2016, ADA4937 Datasheet.
[35] A. Rumiantsev and N. Ridler, “VNA calibration,” IEEE Microwave Magazine, vol. 9, no. 3, pp. 86-99, 2008.
[36] M. Min, O. Martens, and T. Parve, “Lock-in measurement of bio-impedance variations,” Measurement, vol. 27, no. 1, pp. 21-28, 2000.
[37] K. Han, H. Kim, J. Kim, D. You, H. Heo, Y. Kwon, J. Lee, and H. Ko, “A 24.88 nV/VHz wheatstone bridge readout integrated circuit with chopper-stabilized multipath operational amplifier,” Applied Sciences, vol. 10, no. 1, 2020.

In certain embodiments, a particular software module may comprise disparate instructions stored in various locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

In addition, data being tied or rendered together in a database record may be resident in the same memory device, or across several memory devices, and may be linked together in fields of a record in a database across a network. Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMs, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

It will be understood by those having ordinary skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A method for tuning a bioimpedance measurement system, the method comprising the steps of:
generating an input signal;
sending the input signal into a port of a circulator;
applying a load to a port of the circulator; and
tuning the input signal towards a match point of the circulator.

2. The method of claim 1, wherein the circulator comprises an active, quasi-circulator.

3. The method of claim 1, wherein the step of tuning the input signal comprises increasing sensitivity of an output signal to a pulsatile signal.

4. The method of claim 3, wherein the pulsatile signal is generated by a heartbeat.

5. The method of claim 1, wherein the load comprises a portion of a human body.

6. The method of claim 5, wherein the portion of the human body comprises a blood vessel, and wherein the bioimpedance measurement system is non-invasive.

7. The method of claim 1, wherein the step of tuning the input signal towards a match point of the quasi-circulator comprises tuning the input signal to the match point.

8. The method of claim 1, wherein the input signal has a frequency of at least 1 MHz.

9. A method for increasing sensitivity to a pulsatile signal in a bioimpedance measurement system, the method comprising the steps of:
generating an input signal;
sending the input signal into a first port of a circulator;
passing the input signal through a portion of a human body;
following the step of passing the input signal from the circulator through a portion of a human body, outputting the input signal from the circulator; and
tuning the input signal to increase a pulsatile component of an output signal.

10. The method of claim 9, wherein the circulator comprises an active quasi-circulator.

11. The method of claim 10, wherein the step of tuning the input signal comprises adjusting an impedance associated with the input signal towards a match point of the quasi-circulator.

12. The method of claim 11, wherein the match point of the quasi-circulator comprises a point at which a magnitude of the output signal is zero.

13. The method of claim 9, wherein the input signal has a frequency of at least 1 MHz.

14. The method of claim 9, wherein the pulsatile component comprises a heartbeat signal.

15. A system for tuning bioimpedance measurements to increase sensitivity to pulsatile signals, comprising:
an alternating current generator configured to generate an input signal into a human body;
a quasi-circulator configured to receive the input signal from the human body and output an output signal; and
a tuning module configured to adjust a component of the input signal to increase sensitivity of the output signal to pulsatile impedance changes.

16. The system of claim 15, wherein the tuning module is configured to adjust an impedance associated with the input signal.

17. The system of claim 16, wherein the tuning module is configured to digitally adjust the impedance.

18. The system of claim 15, wherein the tuning module is configured to increase sensitivity of the output signal to pulsatile impedance changes by tuning the input signal towards a match point of the quasi-circulator.

19. The system of claim 18, wherein the match point of the quasi-circulator is reached when the output signal of the quasi-circulator is zero.

20. The system of claim 15, wherein the input signal has a frequency of at least 1 MHz.
